SEQUENCE LISTING (4 sequences)

SEQ ID NO: 1 - DNA, 20 bp, Homo sapiens
gaacatgtcccaacatgttg

SEQ ID NO: 2 - DNA, 20 bp, Homo sapiens
caacatgttgggacatgttc

SEQ ID NO: 3 - DNA, 20 bp, Homo sapiens
agactgggcatgtctgggca

SEQ ID NO: 4 - DNA, 20 bp, Homo sapiens
tgcccagacatgcccagtct

(19) United States
(12) Patent Application Publication
(10) Pub. No.: US 2013/0345164 A1
(43) Pub. Date: Dec. 26, 2013

(54) TREATMENTS FOR CELLULAR PROLIFERATIVE DISORDERS AND IDENTIFICATION THEREOF

(71) Applicants: Alexei Vazquez, New Brunswick, NJ (US); Darren Carpizo, New Brunswick, NJ (US); Arnold Levine, Doylestown, PA (US)

(72) Inventors: Alexei Vazquez, New Brunswick, NJ (US); Darren Carpizo, New Brunswick, NJ (US); Arnold Levine, Doylestown, PA (US)

(73) Assignee: UNIVERSITY OF MEDICINE AND DENTISTRY OF NEW JERSEY, Somerset, NJ (US)

(21) Appl. No.: 13/642,613

(22) PCT Filed: Apr. 21, 2011
(86) PCT No.: PCT/US11/33386

§ 371 Date: May 9, 2013

Related U.S. Application Data
(60) Provisional application No. 61/326,490, filed on Apr. 21, 2010.
(60) Provisional application No. 61/438,678, filed on Feb. 2, 2011.

(51) Int. Cl. G06F 19/00; A61K 31/7068; A61K 31/58; A61K 31/4427; A61K 31/519; A61K 31/496; A61K 31/4741; A61K 31/4745; A61K 31/4709; A61K 31/437; A61K 31/4545; A61K 31/4402; A61K 31/4184; A61K 31/407; A61K 31/381; A61K 31/365; A61K 31/282; A61K 31/17; A61K 31/136; A61K 31/473; A61K 31/706

(52) U.S. Cl. CPC G06F 19/34; A61K 31/706; A61K 31/7068; A61K 31/58; A61K 31/4427; A61K 31/519; A61K 31/496; A61K 31/4741; A61K 31/4745; A61K 31/4709; A61K 31/437; A61K 31/4545; A61K 31/4402; A61K 31/4184; A61K 31/407; A61K 31/381; A61K 31/365; A61K 31/282; A61K 31/17; A61K 31/136; A61K 31/473
USPC 514/49; 514/43; 514/173; 514/210.2; 514/249; 514/253.11; 514/264.11; 514/280; 514/283; 514/284; 514/285; 514/316; 514/357; 514/394; 514/413; 514/443; 514/450; 514/492; 514/596; 514/641; 514/656; 702/19

(57) ABSTRACT

This invention concerns methods of identifying treatments for treating various disorders and related computer products and systems. Also disclosed are methods for treating cellular proliferative disorders and use of compounds identified for such treatment.

### CROSS REFERENCE TO RELATED APPLICATIONS

This application claims priority of U.S. Provisional Application No. 61/326,490, filed on Apr. 21, 2010, and U.S. Provisional Application No. 61/438,678, filed on Feb. 2, 2011. The contents of the applications are incorporated herein by reference in their entirety.

### BACKGROUND OF THE INVENTION

There is a need for drugs and reagents for treating various disorders. For example, effective chemotherapy for treating cancer is a continuing goal in the oncology field. Many potential anticancer treatments have been developed. Yet, it has become increasingly clear that specific tumor types respond differently to the many potential treatments.

### SUMMARY OF THE INVENTION

This invention is based, at least in part, on an unexpected discovery of a new methodology for determining whether a test treatment is effective for treating a disorder.

Accordingly, one aspect of this invention features a method for constructing a model for determining whether a test treatment is effective for treating a disorder. In particular, the method is suitable for determining whether a test treatment is effective for treating a disorder that has a specific feature. The method includes steps of:
obtaining data of responsiveness of a panel of cell lines to the test treatment, where the panel comprises or consists of (i) a Case Group consisting of a plurality of case cell lines each of which has a feature characterized of the disorder and (ii) a Control Group consisting of a plurality of control cell lines each of which lacks the feature;
calculating a Responsive Fraction of the case cell lines in the Case Group that are responsive to the test treatment;
calculating a Non-Responsive Fraction of the control cell lines in the Control Group that are not responsive to the test treatment;
generating a Score of the test treatment according to Formula I:

Score=the Responsive Fraction×the Non-Responsive Fraction,
or/and calculating statistical significance, P-value, for enrichment of responsive cell lines in the Case Group relative to the Control group using Fischer's exact test; and
displaying a record comprising the Score or the P-value.
   The value of the Score indicates effectiveness of the test treatment for treating the disorder and the P-value quantifies statistical significance.

In the method, the test treatment can be any treatments, including one selected from the group consisting of a test compound, a microorganism (e.g., a virus), a radiation, a force, a field, a thermal energy, and a lack of a material. Examples of the test compound include, but are not limited to, a small molecule compound, a peptide, a polypeptide, a protein (e.g., an antibody), a nucleic acid, a carbohydrate, or a lipid.

In one embodiment, the test treatment is a test Compoundⱼ and, in that case, the responsiveness of a case cell line or a control cell line in the panel to test Compoundⱼ is obtained by a process having steps of: obtaining an IC50 value of said test Compoundⱼ against each of the cell lines in the panel of cell lines; and calculating a Relative Sensitivity (RS) of said test Compoundⱼ against a Cell Lineᵢ over all of the cell lines in the panel according to Formula II-1:

RSᵢⱼ=log₁₀IC50ᵢⱼ−Average(log₁₀IC50)ⱼ,

In Formula II-1, RSᵢⱼ represents the RS of Compoundⱼ against Cell Lineᵢ; IC50ᵢⱼ represents the log₁₀IC50 of test Compoundⱼ against Cell Lineᵢ; Average(log₁₀IC50)ⱼ represents the average IC50 of test Compoundⱼ against all of the cell lines in the panel, i=1, 2, . . . , n; and j=1, 2, . . . m. Cell Lineᵢ is determined to be (a) responsive to test Compoundⱼ if RSᵢⱼ is less than 2σ, or (b) non-responsive to test Compoundⱼ if RSᵢⱼ is no less than 2σ. The IC50 value is the concentration of a compound that is necessary to inhibit by 50% the growth of treated cells relative to untreated cells; σ represents one standard deviation; 2σ represents two standard deviations.

In another embodiment, the test treatment is ionizing radiation and the responsiveness of a case cell line or a control cell line in the panel to ionizing radiation is obtained by a process having steps of obtaining an ID50 value of the ionizing radiation (i.e., the dose of the radiation that is necessary to inhibit by 50% the growth of treated cells relative to untreated cells) against each of the cell lines in the panel of cell lines; and calculating a Relative Sensitivity (RS) of ionizing radiation against a Cell Lineᵢ in the panel over all of the cell lines in the panel according to Formula II-2:

RSᵢ=ID50ᵢ−Average(ID50),

In Formula II-2, RSᵢ represents the RS of the ionizing radiation against Cell Lineᵢ; ID50ᵢ represents the ID50 of the ionizing radiation against Cell Lineᵢ; Average(ID50) represents the average ID50 of the ionizing radiation against all of the cell lines in the panel, and i=1, 2, . . . , n. The Cell Lineᵢ is determined to be (a) responsive to the ionizing radiation if RSᵢ is less than 2σ, or (b) non-responsive to the ionizing radiation if RSᵢ is no less than 2σ. In certain embodiments, n is at least 2, 3, 5, 10, 15, 20, 30, 50, 60, or 100.

In a second aspect, the invention features a method of determining whether a test treatment is effective for treating a disorder. The method includes steps of obtaining a Score or P-value of the test treatment using the model constructed by the method described above. A Score value for a treatment, if no less than or greater than 0.25 (e.g., 0.25, 0.30, 0.35, 0.40, 0.45, 0.50, or 0.60) indicates that the treatment should be effective in treating the disorder. The P-value, if no larger than or less than 0.05 (e.g., 0.04, 0.03, 0.02, 0.01, 0.005, or, 0.001), also indicates that the test treatment is effective for treating the disorder. For example, the P-value, if no larger than 0.05, or/and the Score, if no less than 0.5, indicates that the test treatment is effective for treating the disorder.

In each of the above-described methods, the record can have one or more of the following values:
(a) a disorder value that identifies the disorder;
(b) a feature value that identifies the feature characterized of the disorder;
(c) a treatment value that identifies the test treatment;
(d) a panel value that identifies the panel of cell lines;
(e) a cell value that identifies each of the cell lines in the panel;
(f) a case group value that identifies the plurality of case cell lines;
(g) a control group value that identifies the plurality of control cell lines;
(h) a responsiveness value that identifies the responsiveness of each of the cell lines in the panel to the test treatment;
(i) a score value that identifies the Score of the test treatment; and
(j) a significance value that identifies the P-value of the test treatment.
   The responsiveness value can identify the RS of a Cell Lineᵢ to a Test Treatmentⱼ (RSᵢⱼ), where i=1, 2 . . . n, and ⱼ=1, 2 . . . m.

In one embodiment, the disorder is a cellular proliferative disorder. A cellular proliferative disorder refers to a disorder characterized by uncontrolled, autonomous cell growth, including malignant and non-malignant growth. Examples include various cancers. The feature of the disorder can be a mutation in a gene, a gene copy alteration, overexpression or loss of a cellular gene, an alteration in a signal transduction pathway, or a resistance to a drug. In one example, the gene is an oncogene or a tumor suppressor gene, including, but not limited to, the BRAF gene, the p53 gene, the PTEN gene, or the RAS gene.

In a third aspect, the invention features a machine-readable medium for carrying out the methods described above. The machine-readable medium has machine-readable instructions encoded thereon which, when executed by a processor, cause a machine having or linked to the processor to execute each of the methods. This invention also features a computer system having the machine-readable medium and a user interface capable of receiving the above-mentioned data and displaying the above-mentioned record.

In a fourth aspect, the invention features a machine-readable medium on which is stored a database capable of configuring a computer to respond to queries based on a plurality of records or values belonging to the database. Each of the records has one or more of the following values:
(a) a disorder value that identifies a disorder;
(b) a feature value that identifies a feature characterized of the disorder;
(c) a treatment value that identifies a test treatment for the disorder;
(d) a panel value that identifies a panel of cell lines;
(e) a cell value that identifies each of the cell lines in the panel;
(f) a case group value that identifies a plurality of case cell lines that have the feature;
(g) a control group value that identifies a plurality of control cell lines that lack the feature;
(h) a responsiveness value that identifies responsiveness of each of the cell lines in the panel to the test treatment;
(i) a score value that identifies a Score of the test treatment; and
(j) a significance value that identifies a P-value of the test treatment.
   The responsiveness value can identify the RS of a Cell Lineᵢ to a Test Treatmentᵢⱼ, RSᵢⱼ, where i=1, 2, . . . n, and j=1, 2, . . . m. The invention also features a computer system having the machine-readable medium and a user interface capable of (a) receiving a selection of one or more values for the test treatment or the cell line (e.g., values (a)-(g) listed above) for determining a match between the values and a responsiveness value in the database, and (b) displaying a record associated with a matching responsiveness value.

In any of the above-mentioned machine-readable media, the test treatment is selected from the group consisting of a test compound, a microorganism, a radiation, a force, a field, a thermal energy, and a lack of a material. In any of the above-mentioned computer systems, the user interface is capable of displaying the record in the format of a relative responding histogram (RRH) or of a response graph (RG). In this invention, the above-mentioned n or m can be at least 2, 3, 5, 10, 15, 20, 30, 50, 60, or 100 in certain embodiments.

In a fifth aspect, the invention features a method for treating a cellular proliferative disorder in a subject. The method includes administering to a subject in need thereof an effective amount of a compound selected from the group consisting of those compounds in the four sub-groups listed below or a pharmaceutically acceptable salt of the compound.

1. Compounds specific for disorders characterized by a mutation in the p53 gene: NSC319726, NSC319725, NSC328784, NSC612941, NSC155694, NSC694266, and NSC93739.
2. Compounds specific for disorders characterized by a V600E mutation in the BRAF gene: NSC656238, NSC682449, NSC690432, NSC741078, NSC706829, NSC669995, NSC361127, NSC263637, and NSC354462.
3. Compounds specific for disorders characterized by a mutation in the KRAS gene: NSC613327, NSC146268, NSC740, NSC696558, NSC666787, NSC682306, NSC117356, NSC739, NSC680417, NSC363981, and NSC266046.
4. Compounds specific for disorders characterized by a mutation in the PTEN gene: NSC706744, NSC735493, NSC734294, NSC681640, NSC681645, NSC681634, NSC681638, NSC606499, NSC606498, NSC606497, NSC364830, NSC639174, NSC620256, NSC363979, NSC363980, NSC363981, NSC378734, NSC378735, NSC378727, NSC355447, NSC368891, and NSC48006.

In a preferred embodiment, the cellular proliferative disorder is characterized by a mutation in a gene, such as the p53 gene, the RAS gene, the BRAF gene, or the PTEN gene. In one example, the cellular proliferative disorder is characterized by a mutation in the p53 gene and the compound is selected from the group consisting of those in Subgroup 1. In another example, the cellular proliferative disorder is characterized by a mutation in the BRAF gene and the compound is selected from the group consisting of those in Subgroup 2. In a third example, the cellular proliferative disorder is characterized by a mutation in the KRAS gene and the compound is selected from the group consisting of those in Subgroup 3. In a further example, the cellular proliferative disorder is characterized by a mutation in the PTEN gene and the compound is selected from the group consisting of those in Subgroup 4.

The invention also features use of a compound selected from the group consisting of NSC669995, NSC682449, NSC656238, NSC612941, NSC155694, NSC319726, NSC319725, NSC694266, and NSC93739, or a pharmaceutically acceptable salt of the compound, for the treatment of a cellular proliferative disorder. The invention further features use of a compound selected from the group consisting of NSC669995, NSC682449, NSC656238, NSC612941, NSC155694, NSC319726, NSC319725, NSC694266, and NSC93739, or a pharmaceutically acceptable salt of the compound, in the manufacture of a medicament for the treatment of a cellular proliferative disorder.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a flowchart showing operations in an example of identifying compounds for treating a disorder.

FIG. 2 is a diagram showing a Relative Responding Histogram (RRH).

FIG. 3 is a diagram showing a Response Graph (RG).

FIGS. 4a and 4b are a set of diagrams showing structures of six compounds that were identified as candidates for treating cancer and GI50s of the six compounds against various tumor cell lines.

FIGS. 5a-b are a set of diagrams showing (a) chemical identities and structure scheme of two thiosemicarbazone family compounds, NSC319725 and NSC319726, identified by a method disclosed in the application, (b) clustering of cell lines with sensitivity to the two compounds, based on computational analysis of the NCI60 database, and (c) validation of the two compounds in mouse embryonic fibroblast (MEF) cells with individual p53 mutations, using 96-well MTS assay.

FIG. 6 is a diagram showing an exemplary computer system.

FIGS. 7a-c are a set of diagrams showing allele specificity of p53 R175 mutations to NSC319726, which is demonstrated with (a) sensitivity to NSC319726 of MEF cells with endogenous p53R172H mutation (equivalent to human p53R175H mutation), (b) sensitivity to of NSC319726 of human cells with wild type p53 (H460, HCT116, MCF7 and WI38) and in p53-null cell line (SKOV3), and (c) sensitivity to NSC319726 of human tumor cells with various p53 mutations.

FIGS. 8a-c are a set of diagrams showing induction of p53R175 mutation-dependent apoptosis by NSC319726 in MEF cells with exogenous p53 mutations (b) and in three human ovarian carcinoma cell lines with p53 mutations (TOV112D (p53R175H), OVCAR3 (p53R248W) and SKOV3 (p53-null)) (b), and p53-dependent growth inhibition in TOV112D cells where siRNA to p53 abrogated the growth inhibition by NSC319726 (c).

FIGS. 9a-c are a set of photographs and diagrams showing conformational change induced by NSC319726, but not NSC319725, in p53R175H mutant protein.

FIGS. 10a-e are a set of photographs and diagrams showing functional recovery of the p53 protein upon NSC319726 treatment: (a) Induction of p21 protein in TOV112D cells but not in SKOV3 upon NSC319726 treatment, shown in Western blot; (b) Induction of the p53 mutant protein binding to the 20-bp, p53-recognition element (p53RE) in the p21 promoter region, shown in luciferase activity assay; (c) Induction of the p53-target genes (p21, PUMA and MDM2) as shown in quantitative real-time PCR; (d) Microarray analysis of the p53 target genes in the TOV112D cells with treatment of NSC319725 and NSC319726. Each treatment had three independent repeats (labeled as 1, 2, and 3); (e) Comparison of the p53 expression signatures of 319726 treated cells with that of p53 wild type, p53 null and p53R175H cell lines in response to gamma-irradiation.

FIGS. 11 a-c are a set of diagrams, a set of photographs, and a table showing in-vivo evidence of p53 mutant synthetic lethality in response to 319726 treatment: a) Toxicity assays performed in p53 wild type, p53 null, p53^{+/R172H} and p53^{R172H/R172H} mice indicating dependence of toxicity on TP53R175H expression; b) Cleaved caspase-3 immunostaining in spleen and thymus tissues of NSC319726 treated p53 wild type and p53^{R172H/R172H} mice demonstrating increased apoptosis in the p53^{R172H/R172H} mice, and c) Comparative expression of p53 targets by quantitative PCR across various tissues of NSC319726 treated p53 wild type and p53^{R172H/R172H} mice indicating significant upregulation in several tissues of the p53^{R172H/R172H} mice.

FIG. 12 is a set of diagrams showing that compound NSC319726 treatment inhibited mouse xenograft tumor growth in an allele specific mutant p53 dependent manner. Tumor cell lines examined included (a) H460-p53 wildtype (n=1), (b) MDAMB468-p53^{R273H} (n=1), (c) HCT116-p53 null (n=1) and (d) TOV112D-p53^{R175H} (n=7).

FIG. 13 is a set of diagrams showing chemical identities and structure scheme of 3 compounds, NSC319726, NSC319725, and NSC328784, identified as being specific for disorders characterized by a mutation in the p53 gene and related clustering of cell lines with sensitivity to the compounds based on computational analysis of the NCI60 database.

FIG. 14 is a set of diagrams showing chemical identities and structure scheme of 9 compounds identified as being specific for disorders characterized by a V600E mutation in the BRAF gene and related clustering of cell lines with sensitivity to the compounds based on computational analysis of the NCI60 database.

FIG. 15 is a set of diagrams showing chemical identities and structure scheme of 11 compounds identified as being specific for disorders characterized by a mutation in the KRAS gene and related clustering of cell lines with sensitivity to the compounds based on computational analysis of the NCI60 database.

FIG. 16 is a set of diagrams showing chemical identities and structure scheme of 19 compounds identified as being specific for disorders characterized by a mutation in the PTEN gene and related clustering of cell lines with sensitivity to the compounds based on computational analysis of the NCI60 database.

### DETAILED DESCRIPTION OF THE INVENTION

This invention relates to methods and systems for identifying candidate treatments for treating various disorders, e.g., treatments for cancer with increased activity in specific tumor types. The invention also relates to compositions and methods for treating cellular proliferative disorders.

### Methodology

In one aspect, this application provides a highly efficient analysis methodology for identifying candidate treatments for treating various disorders. The methodology is based, at least in part, on a rigorous statistical definition of good response and the use of a Score function to rank test treatments (e.g., compounds) according to their selective activity in a specific group of disease cell lines (e.g., tumor-derived cell lines). The methodology involves processing multi-dimensional input raw data sets across a large number of studies and experiments from diverse technologies as well as different biological and chemical assays, data types, and organisms. For example, the above-mentioned data of responsiveness includes one or more values identifying, among others, a disorder, a test treatment, a panel of cell lines, each of the cell lines in the panel, responsiveness of each of the cell lines in the panel to the test treatment, a feature characterized of the disorder, the plurality of case cell lines/Case Group, and the plurality of control cell lines/Control Group. The methodology allows one to combine orthogonal types of data and available public knowledge to identify treatments for various disorders, such as cellular proliferative disorders.

In one example, application of this methodology proceeds by, first, identifying a group of cell lines representing the specified tumor type and an appropriate control, and, second, ranking the treatments in the screen according to their specific anticancer activity in one of the groups but not in the other. In one embodiment, the input multi-dimensional raw data sets include responsiveness of a panel of tumor derived cell lines and control/normal cell lines to a panel of potential anticancer treatments. The responsiveness can be measured using different protocols, including but not limited to, growth inhibition assays.

Data from individual tumors/cell lines or treatments may be correlated with other orthogonal data and public information, e.g., that available from National Cancer Institute (NCI), such as the NCI60 screen data, which reported the IC50s of 47,624 compounds against 60 tumor derived cell lines (NCI60 screen, October 2009 release, which is available at dtp.nci.nih.gov/docs/cancer/cancer_data.html).

As more of such data becomes publicly available, the data can be correlated with previous findings on relevant cell lines, gene mutations, and treatments. For example, the multi-dimensional data sets can include data of tumor cell lines, related gene mutations, related gene methylations, and related chromosomal aberrations. The multi-dimensional data sets can include data from studies of various new treatments on the cells or related tumors. The methodology disclosed in this application allows the data sets to be combined and used to elucidate tumors' sensitivity or resistance to the treatments.

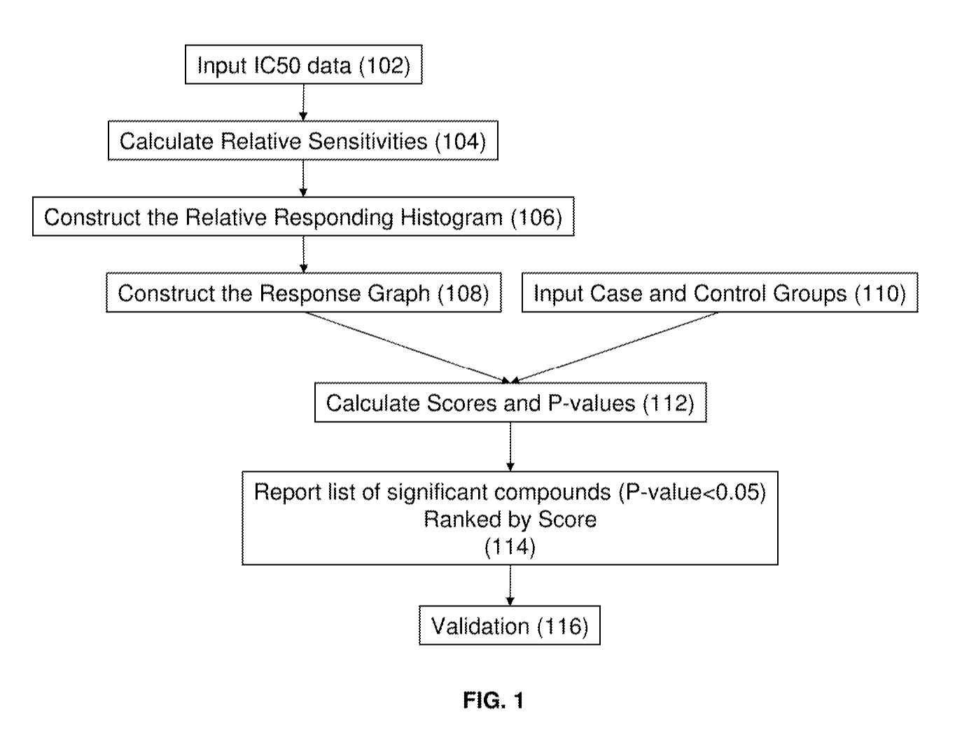

Shown in FIG. 1 is an exemplary process flow diagram showing key operations in the methodology disclosed in this application for identifying effective test compounds. The process generates a Score indicating the efficacy of a panel of treatments (e.g., test compounds) on a panel of cell lines.

First, input IC50 data are received (102). Each data point represents the IC50 value of a test compound against each of the cell lines in the panel of cell lines. The IC50 values can be obtained via assays known in the art. Alternatively, one can obtain such data from publicly available databases, such as that maintained as NCI, in the manner shown in the working example below.

Then, responsiveness data of the cell lines to the compounds are then calculated (104). For example, a Relative Sensitivity (RS) of a test Treatmentⱼ (e.g., Compoundⱼ) against a Cell Lineᵢ over all of the cell lines in the panel can be calculated according to Formula II-1 mentioned above:

RSᵢⱼ=log₁₀IC50ᵢⱼ−Average(log₁₀IC50)ⱼ.

Generally, the data is given by a matrix reporting the response sensitivity of a panel of tumor-derived cell lines to a panel of test treatments. This matrix is transformed into Relative Sensitivities (RSes), by reporting the sensitivity measurement for each treatment relative to its average value across the entire panel of cell lines. For each pair of treatment and cell line, one obtains a value quantifying the relative response sensitivity of that cell line to that treatment.

In Formula II-1, RSᵢⱼ represents the RS of Treatmentⱼ (e.g., Compoundⱼ) against Cell Lineᵢ: IC50ᵢⱼ represents the IC50 of Treatmentⱼ (e.g., Compoundⱼ) against Cell Lineᵢ; and Average(log₁₀IC50)ⱼ represents the average IC50 of Treatmentⱼ (e.g., Compoundⱼ) against all of the cell lines in the panel. The variables “i” and “j” are used to designate the cell lines and the treatments (e.g., compounds), respectively, where i=1, 2, . . . , n; and j=1, 2, . . . , m. Cell Lineᵢ is determined (a) to be responsive (or have a good response) to Treatmentⱼ if RSᵢⱼ is less than two standard deviations (i.e., −2σ), or (b) to be non-responsive (or have a bad response) if RSᵢⱼ is no less than two standard deviations.

Once RSes are calculated, one can evaluate the relative sensitivities for all pairs of treatment-cell line by constructing a Relative Responding Histogram (RRH), a histogram of relative response sensitivities across all treatments and cell lines (106). RRH is informative about whether the right variable is being used to quantify the response sensitivity. Ideally the RRH should follow an approximately symmetric distribution, centered at zero and with fast decaying left and right tails. In such an ideal scenario, good responses can be clearly distinguished from bad responses, as both are located at the opposite tails with the typical or expected behavior in between. If this is not the case, some transformation should be applied to the sensitivity measurement such that the resulting RRH satisfy those desired properties. For example, for small molecule compounds, a typical response sensitivity measurement is the IC50, the concentration necessary to inhibit the growth of treated cells 50% relative to untreated controls. The histogram of the IC50s is, however, often squeezed towards the zero concentration and has a right tail only. This is because some cell lines are highly insensitive to treatments of some small molecules, with IC50s above the micro molar range. Thus, when plotting all measurements together, responses between nano- and micro-molar IC50s are practically indistinguishable. A way around this problem is to work with the log₁₀IC50 and define the relative sensitivity as RS=log₁₀IC50-average(log₁₀IC50) as shown above. With the RSes defined in this way, a symmetric RRH is obtained, centered at zero, with a left tail for the good responses (low IC50) and a right tail for the bad responses (high IC50s) (Vazquez, A., BMC Syst Biol, 2009. 3: p. 81). In the following it is assumed that the good and bad responses are located at the left and the right tails, respectively.

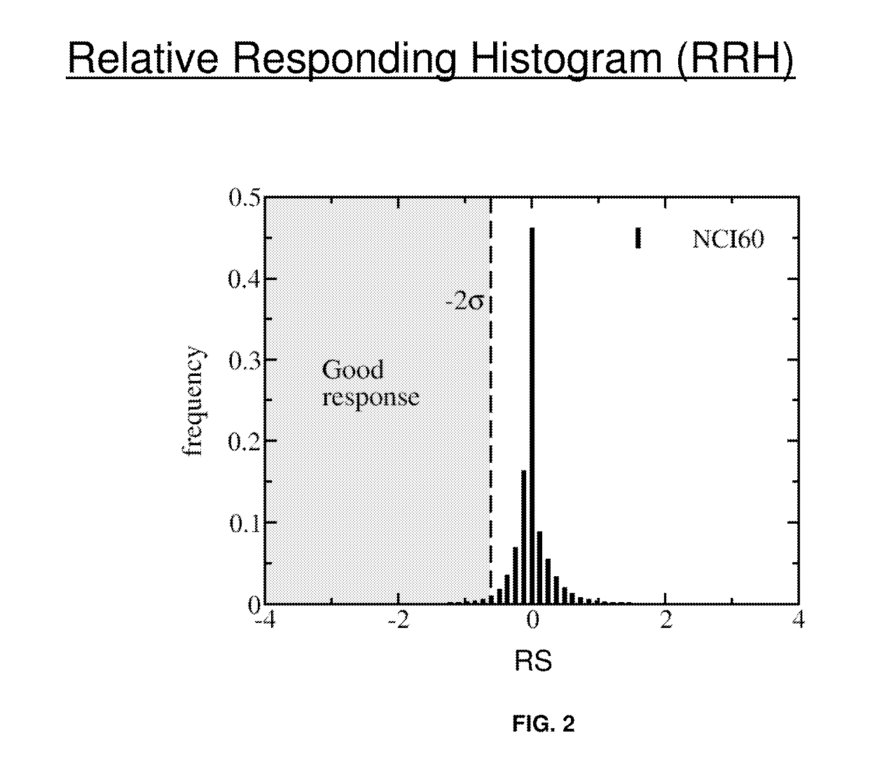

Shown in FIG. 2 is an exemplary RRH, illustrating statistical identification of good responses, located at the left tail of the RRH. As mentioned above, RSes below two standard deviations on the left tail (−2σ) are defined as good responses. Thus, statistically speaking, when a cell line is said to be a good responder to a treatment, it means that, with about a 95% confidence, this cell line is indeed sensitive to that treatment (e.g., the IC50 is low). It is worth noticing that, in analogy with the definition of good responders, the right tail of the RRH can be used to define the resistance cell lines, which will become useful for some of the applications listed below.

In addition to constructing RRH, one can also construct a Response Graph (RG) to evaluate and visualize the relative sensitivities for all pairs of treatments and cell lines (108). Using the definition of good response provided above, the treatment-cell line response graph (RG) is constructed with a class of nodes representing treatments, another class of nodes representing cell lines, and a line connecting a treatment to a cell lines whenever the latter has a good response to the former (Vazquez, A., BMC Syst Biol, 2009. 3: p. 81). The RG represents a discrete version of the response sensitivity matrix. It has the advantage of distinguishing good responses from the rest of the data.

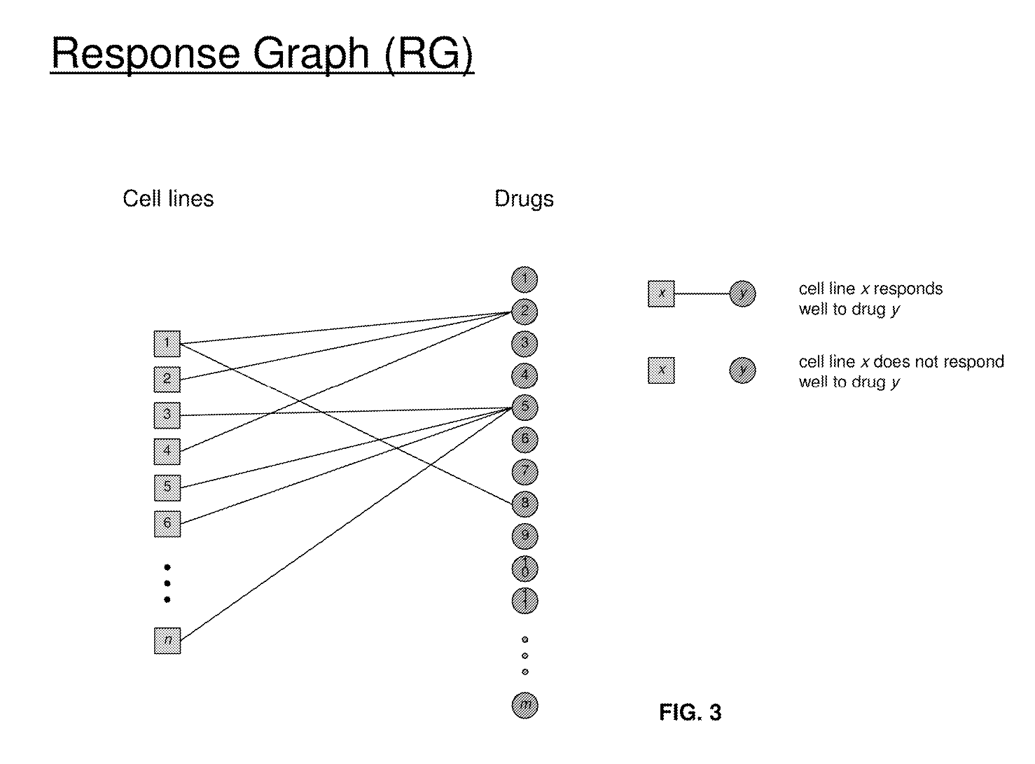

Shown in FIG. 3 is an exemplary RG. In the RG, a line linking a cell line and a treatment (e.g., a drug) indicates the cell line is responsive to the treatment. For example, in FIG. 3, a line linking Cell Line 1 and Drug 8 indicates that Cell Line 1 responds to Drug 8. By the same token, Cell Lines 1, 2, and 4 are responsive to Drug 2 while Cell Lines 3, 5, and 6 are responsive to Drug 5. Conversely, an absence of a line between a cell line and a drug indicates that the cell line does not respond the drug. For example, Cell line 1 does not respond to Drug 1, 3, or 4).

To use the methodology described herein for determining whether a test treatment (e.g., a test compound) is effective for treating a disorder, one can obtain data of responsiveness of a panel of cell lines for that disorder to a test treatment. Specifically, one can divide the panel into two groups based on whether a feature characterized of the disorder is shared by various cell lines: (1) a Case Group consisting of a plurality of case cell lines each of which has a feature characterized of the disorder and (2) a Control Group consisting of a plurality of control cell lines each of which lacks the feature (110).

One can then calculate Scores and P-values for the compounds as shown in FIG. 1 (112). The Score calculations are carried out by:
(1) calculating a Responsive Fraction, i.e., the fraction of the case cell lines in the Case Group that are responsive to the test treatment;
(2) calculating a Non-Responsive Fraction i.e., the fraction of the control cell lines in the Control Group that are not responsive to the test treatment; and
(3) generating a Score of the test treatment according to Formula I:

Score=the Responsive Fraction×the Non-Responsive Fraction.

In other words, the Score is the product of the fraction of good responders in the Case Group and the fraction of non-good responders in the Control Group. This Score function ranks higher those treatments with an enrichment of good responders in the Case Group, while simultaneously having a depletion of good responders (thus an enrichment of not good responders) in the Control Group. The statistical significance, P-value, for enrichment of responsive cell lines in the Case Group relative to the Control Group can be obtained using Fischer's exact test.

The value of the Score of a test treatment indicates effectiveness of the test treatment for treating the disorder and the P-value quantifies statistical significance. For example, a Score value for a treatment, if no less than or greater than 0.25 (e.g., 0.25, 0.30, 0.35, 0.40, 0.45, 0.50, or 0.60) indicates that the treatment should be effective in treating the disorder. The P-value, if no larger than 0.05 (e.g., 0.04, 0.03, 0.02, 0.01, 0.005, or, 0.001), also indicates that the test treatment is effective for treating the disorder. Scores for all the treatments can be obtained in the same way and ranked according to their values. In a preferred embodiment, the Scores of all treatments are ranked, starting at 1, in a decreasing order (114). If necessary, a validation process (116) can be further conducted.

As a proof of principles, compounds with specific activity against tumor cell lines carrying a mutation in the tumor suppressor gene p53 or the oncogene BRAF were evaluated using the methodology disclosed herein and their actual specificity demonstrated. As shown in the examples below, the methodology successfully identified effective compounds. Accordingly, the methodology can be used for identifying a cancer treatment having a better response for a specific cell type which comprises identifying both a group of cell lines representing the specified cell type and a group of cell lines which constitute an appropriate control group and ranking an anticancer compound according to its selective activity in the specific group of derived cell lines and its inactivity in the control group. The methodology can accelerate the process of discovery of treatments for personalized anticancer treatment.

The methodology disclosed in this application has a wide range of applications. In addition to search of treatments with specific activity in cells carrying a mutation in B-Raf and p53, this methodology can be applied to identify candidate compounds with an increased activity in tumors carrying other somatic mutations in cancer related genes. In particular, this is useful for somatic mutations with high prevalence in tumors. Examples include mutations in the PTEN and KRAS genes. Within the above-mentioned NCI60 screen, there are 12 cell lines with a PTEN mutation or deletion and 11 cell lines with a KRAS mutation. Thus, this methodology can be applied to find compounds with specific activity in tumors carrying a PTEN or KRAS alteration, using as input the publicly available NCI60 screen data.

The methodology can be also applied to search for treatments with increased activity in tumors with defined alterations, provided there are enough samples representing the case and control groups. Examples of these alterations include, but are not limited to, somatic mutations, gene copy alterations, overexpression or under-expression of gene, or even more complex molecular phenotypes such as pathway alterations, and any combination thereof. This set of alterations can be used to define the case (carrying the alterations) and control (not carrying the alterations) groups.

The methodology can be used to identify treatments (e.g., compounds) for adjuvant therapy. For all available anticancer treatments, a significant number of patients manifest resistance to the treatments. A strategy to overcome this problem is to use an adjuvant therapy that targets those tumors insensitive to the original treatment. A search for adjuvant therapies can be optimized using the methodology disclosed herein. To this end, given a specified treatment, the case group will contain cell lines that are resistant to that treatment (as defined above) while the control group will contain the cell lines that respond well to treatment (as defined above). Taking as input these case and control groups, the methodology disclosed in this application can be used to identify compounds with increased activity in tumors that are resistant to the given treatment, which could be used as adjuvant therapy.

Furthermore, the methodology disclosed herein can be applied to search for treatments with increased activity in any specified tumor group, provided a definition of the case and control groups and enough samples representing them. The input data can be obtained from any anticancer treatment screen, including but not limited to the NCI60 screen. A treatment as used herein refers to any stimulus. Examples include exposure to a compound, such as small molecules (as in the NCI60 screen), peptides, and antibodies, or to others, such as ionizing radiation, or any other entity with anticancer potential. This methodology applies to all such treatments, provided a meaningful definition of relative sensitivity can be obtained.

In the above-described methodology, certain raw data for a disorder and related cell lines are needed. In one embodiment, the data include genetic features, such as gene point mutations, SNP patterns (e.g., haplotype blocks), portions of genes (e.g., exons/introns or regulatory motifs), regions of a genome or chromosome spanning more than one gene. Other examples include phenotypic features such as the morphology of cells and cellular organelles (e.g., cytoskeleton) or their behaviors (e.g., drug-resistance, proliferation, differentiation, cell death, and metastasis). Examples of technology for producing such raw data include, but are not limited to, microarray platforms including RNA and miRNA expression, SNP genotyping, protein expression, protein-DNA interaction and methylation data and amplification/deletion of chromosomal regions platforms, quantitative polymerase chain reaction gene expression platforms, identified novel genetic variants, copy-number variation detection platforms, detecting chromosomal aberrations (amplifications/deletions) and whole genome sequencing. While the description here concerns genetic mutation data, the methods described may be extrapolated to other types of data, e.g., protein sequences and phenotypic features.

The methodology described in this application involves biological experiments in which a stimulus (i.e., a test treatment) acts on a biological sample such as a tissue or cell culture. Often the biological experiment will have associated clinical parameters such as tumor stage, patient history, etc. The sample may be exposed to one or more stimuli or treatments to produce test data. Control data may also be produced in the same way. The stimulus is chosen as appropriate for the particular study undertaken. Examples of stimuli that may be employed can be an exposure to particular materials or compositions, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, peptides, polypeptides, proteins (e.g., antibodies), lipids, microorganisms (e.g., viruses, including those useful in gene therapy), various infectious agents, mixtures of the foregoing, and the like. Other examples of stimuli include the lack of a particular material e.g., a nutrient or a growth factor.

An “antibody” includes intact molecules as well as fragments thereof, such as Fab, F(ab′)2, Fv, scFv (single chain antibody), and dAb (domain antibody). A derivative of an antibody refers to a protein or a protein complex having a polypeptide variant of this invention. An antibody or derivative in this invention can be made by co-expressing corresponding light and heavy chain CDRs-containing polypeptides in a suitable host cell by methods known in the art. See, e.g., Harlow and Lane, (1988) Antibodies: A Laboratory Manual, Cold Spring Harbor Laboratory, New York.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. Examples include an antisense nucleic acid and an RNAi agent.

In a preferred embodiment, the stimulus is an exposure to therapeutic agents (including agents suspected of being therapeutic but not yet proven to have this property). Often the therapeutic agent is a chemical compound such as a drug or drug candidate or a compound present in the environment. The biological impact of chemical compounds is manifest as a change in a feature such as a level gene expression or a phenotypic characteristic, including cell growth, cell proliferation, cell differentiation, and cell death.

### Computer Products, Systems, and Instruments

The methodology of this invention can be incorporated into a multiplicity of suitable computer products, systems, and/or information instruments. User interface methods known in the information processing art can be used in the systems of this invention.

1. Computer Software

For example, the above-disclosed methodology or components thereof can be embodied in a fixed or non-transitory medium (e.g., a computer accessible/computer readable medium program component containing logic instructions or data, or both), that when loaded into an appropriately configured computing device can cause that device to perform operations to the invention. In various embodiments a fixed medium component containing logic instructions can be delivered to a viewer on a fixed medium for physically loading into a viewer's computer or a fixed medium containing logic instructions can reside on a remote server that a viewer can access through a communication medium in order to download a program component.

Examples of a tangible computer-readable medium suitable for use computer program products and computational apparatus of this invention include, but are not limited to, magnetic media such as hard disks, floppy disks, and magnetic tape; optical media such as CD-ROM disks; magneto-optical media; semiconductor memory devices (e.g., flash memory), and hardware devices that are specially configured to store and perform program instructions, such as read-only memory devices (ROM) and random access memory (RAM) and sometimes application-specific integrated circuits (ASICs), programmable logic devices (PLDs) and signal transmission media for delivering computer-readable instructions, such as local area networks, wide area networks, and the Internet. The data and program instructions provided herein may also be embodied on a carrier wave or other transport medium (including electronic or optically conductive pathways). The data and program instructions of this invention may also be embodied on a carrier wave or other transport medium (e.g., optical lines, electrical lines, and/or airwaves).

2. Database

The above-described raw data sets and information generated using the methodology of this invention can be used to establish a database, i.e., a collection of data, which can be used to analyze and respond to queries. In one embodiment, the database includes one or more records for organizing the raw data sets and information sets in a particular hierarchy or directory (e.g., a hierarchy of studies and projects). In addition, the database may include information correlating the records to one another, a list of globally unique terms or identifiers for cancers, tumors, cell lines, genes, treatments (e.g., compounds), or other features. Such a database also contains a taxonomy that contains a list of all tags (keywords) for different tissues, disease states, treatment types, phenotypes, cells, as well as their relationships.

In one embodiment, the database contains data from a number of sources, including data from external sources, such as public databases, including those maintained at the National Cancer Institute and the National Center for Biotechnology Information (NCBI). In addition, the database can include proprietary data obtained and processed by the database developer or user. A database may be updated by a developer or user as new public or private information from biological or chemical experiments becomes available. Once imported, all data are correlated with other information in the database so as to enable users to interrogate tumors, cell lines, biological features, compounds, and responsiveness thereto across the entire information space.

3. Computer Hardware

In another aspect, the invention provides an apparatus for performing the above-mentioned operations. This apparatus may be specially designed and/or constructed for the required purposes, or it may be a general-purpose computer selectively configured by one or more computer programs and/or data structures stored in or otherwise made available to the computer. The processes presented herein are not inherently related to any particular computer or other apparatus. In particular, various general-purpose machines may be used with programs written in accordance with the teachings herein, or it may be more convenient to construct a more specialized apparatus to perform the required method steps.

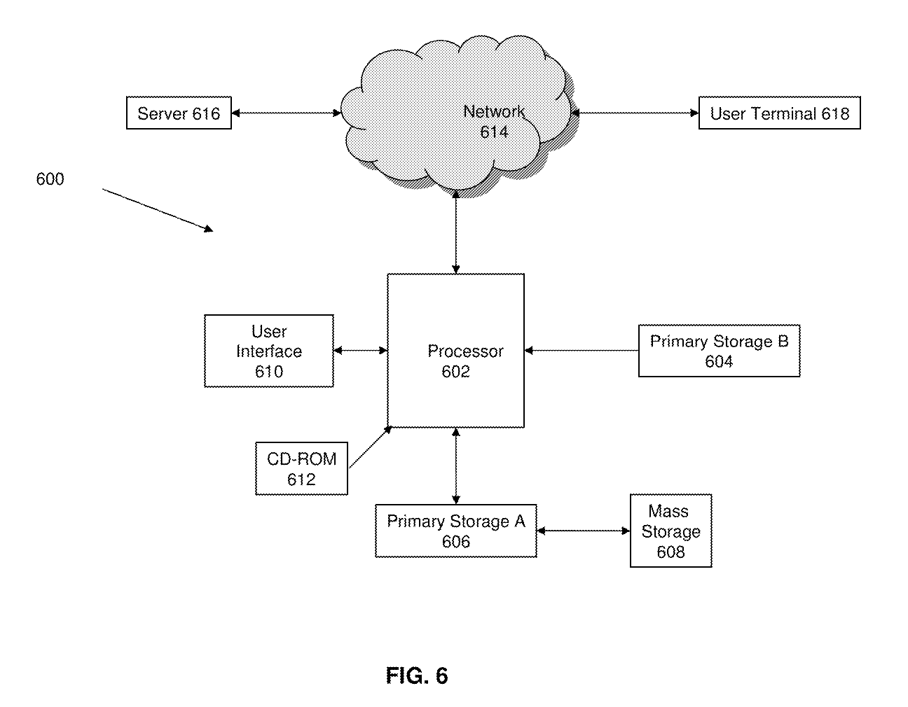

FIG. 6 illustrates an exemplary computer system (600) that, when appropriately configured or designed, can serve as a computational apparatus according to certain embodiments. The computer system 600 includes any number of processors 602 (i.e., central processing units, or CPUs) that are coupled to storage devices including primary storage 606 (typically a random access memory, or RAM), primary storage 604 (typically a read only memory, or ROM). CPU 602 may be of various types including microcontrollers and microprocessors such as programmable devices (e.g., CPLDs and FPGAs) and non-programmable devices such as gate array ASICs or general-purpose microprocessors. In the depicted embodiment, primary storage 604 acts to transfer data and instructions uni-directionally to the CPU and primary storage 606 is used typically to transfer data and instructions in a bi-directional manner. Both of these primary storage devices may include any suitable computer-readable media such as those described above. A mass storage device 608 is also coupled bi-directionally to primary storage 606 and provides additional data storage capacity and may include any of the computer-readable media described above. Mass storage device 608 may be used to store programs, data and the like and is typically a secondary storage medium such as a hard disk. Frequently, such programs, data and the like are temporarily copied to primary memory 606 for execution on CPU 602. The information retained within the mass storage device 608, may, in appropriate cases, be incorporated in standard fashion as part of primary storage 604. A specific mass storage device such as a CD-ROM 612 may also pass data uni-directionally to the CPU or primary storage.

CPU 602 is also coupled to an interface 610 that connects to one or more input/output devices such as video monitors, track balls, mice, keyboards, microphones, touch-sensitive displays, transducer card readers, magnetic or paper tape readers, tablets, styluses, voice or handwriting recognition peripherals, USB ports, or other well-known input devices such as other computers. Finally, CPU 602 optionally may be coupled to an external device such as a database or a computer or telecommunications network using an external connection as shown generally at network 614. With such a connection, it is contemplated that the CPU might receive information from the network, or might output information to the network in the course of performing the method steps described herein.

In one embodiment, a system such as computer system 600 is used as a special purpose data import, data correlation, and querying system capable of performing some or all of the tasks described herein. System 600 may also serve as various other tools associated with database described above and querying such as a data capture tool. Information and programs, including data files can be provided via a network connection 614 for access or downloading from server system 616. Alternatively, such information, programs and files can be provided to the researcher on a storage device. In a specific embodiment, the computer system 600 is directly coupled to a data acquisition system such as a high-throughput screening system that captures data from samples. Data from such systems are provided via interface 610 for analysis by system 600. Alternatively, the data processed by system 600 are provided from a data storage source such as a database or other repository of relevant data. Once in apparatus 600, a memory device such as primary storage 606 or mass storage 608 buffers or stores, at least temporarily, relevant data. The memory may also store various routines and/or programs for importing, analyzing and presenting the data, including importing the above-described data sets, correlating data sets with one another and with feature groups, generating and running queries, etc.

In certain embodiments, the system of this invention may include one or more user terminals (618). User terminals can include any type of computer (e.g., desktop, laptop, tablet, etc.), media computing platforms (e.g., cable, satellite set top boxes, digital video recorders, etc.), handheld computing devices (e.g., PDAs, e-mail clients, etc.), cell phones or any other type of computing or communication platforms. A server (616) in communication with a user terminal may include a server device or decentralized server devices, and may include mainframe computers, mini computers, super computers, personal computers, or combinations thereof. A plurality of server systems may also be used without departing from the scope of the present invention. User terminals and a server system may communicate with each other through the network 614. The network may comprise, e.g., wired networks such as LANs (local area networks), WANs (wide area networks), MANs (metropolitan area networks), ISDNs (Intergrated Service Digital Networks), etc. as well as wireless networks such as wireless LANs, CDMA, Bluetooth, and satellite communication networks, etc. without limiting the scope of the present invention.

### Compositions

Within the scope of this invention is a composition that contains a suitable carrier and one or more of the compounds described above, such as NSC669995, NSC682449, NSC656238, NSC612941, NSC155694, NSC319726, NSC319725, NSC694266, and NSC93739, or a pharmaceutically acceptable salt of the compound.

The composition can be a pharmaceutical composition that contains a pharmaceutically acceptable carrier, a dietary composition that contains a dietarily acceptable suitable carrier, or a cosmetic composition that contains a cosmetically acceptable carrier.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and, preferably, capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, sodium lauryl sulfate, and D&C Yellow #10.

The above-described composition, in any of the forms described above, can be used for treating cellular proliferative disorders. An effective amount refers to the amount of an active compound that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the present invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally comprise one or more excipients or diluents. In some preferred embodiments, the topical formulations include a material that would enhance absorption or penetration of the active agent(s) through the skin or other affected areas. The topical composition is useful for treating cellular proliferative disorders in the skin, such as melanoma.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, allergic response, and the like. The carrier enables an active agent and optional component to be delivered to the skin at an appropriate concentration(s). The carrier can thus act as a diluent, dispersant, solvent, or the like to ensure that the active materials are applied to and distributed evenly over the selected target at an appropriate concentration. The carrier can be solid, semi-solid, or liquid. Preferably, it is in the form of a lotion, a cream, or a gel, in particular one that has a sufficient thickness or yield point to prevent the active materials from sedimenting. The carrier can be inert or possess dermatological benefits. It should also be physically and chemically compatible with the active components described herein, and should not unduly impair stability, efficacy, or other use benefits associated with the composition.

### Treatment Methods

The invention also features methods for treating in a subject a cellular proliferative disorder (e.g., cancer). A cellular proliferative disorder refers to a disorder characterized by uncontrolled, autonomous cell growth, including malignant and non-malignant growth. Examples of this disorder include colon cancer or colorectal cancer, breast cancer, prostate cancer, hepatocellular carcinoma, melanoma, lung cancer, glioblastoma, brain or CNS tumor, hematopoeitic malignancies, leukemia, retinoblastoma, renal cell carcinoma, head and neck cancer, cervical cancer, pancreatic cancer, esophageal cancer, ovarian cancer, and squama cell carcinoma.

In one embodiment, the present invention provides a method for treating a cellular proliferative disorder in a subject, characterized by a mutation in the p53 gene, comprising administering to the subject in need thereof an effective amount of a compound of Formula (Ia) or (Ib):

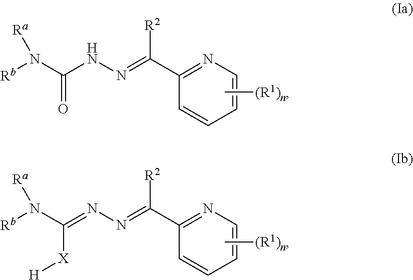

or a pharmaceutically acceptable salt of the compound, wherein:
n is 0, 1, 2, 3, or 4;
X is O, S, or Se;
R¹ at each occurrence is independently C₁-C₄ alkyl, C₁-C₄ alkoxy, hydroxyl, halogen;
R² is H, C₁-C₆ alkyl, or arylalkyl;
R^{a} and R^{b} are each independently selected from the group consisting of hydrogen, C₁-C₆ alkyl, aryl, arylalkyl, or alternatively, R^{a} and R^{b}, together with the N atom to which they are attached, form a heterocyclyl, said heterocyclyl optionally substituted by one to three substituents independently selected from C₁-C₄ alkyl, C₁-C₄ alkoxy, hydroxyl, halogen, aryl, and heteroaryl.

In another embodiment, the present invention provides a method for treating a cellular proliferative disorder in a subject, characterized by a mutation in the p53 gene, comprising administering to the subject in need thereof an effective amount of a compound of Formula (Ia) or (Ib), wherein:
n is 0;
R² is C₁-C₄ alkyl;
X is S or Se;
R^{a} and R^{b} are each hydrogen or C₁-C₄ alkyl, or alternatively, R^{a}R^{b}N— is heterocyclyl selected from the group consisting of:

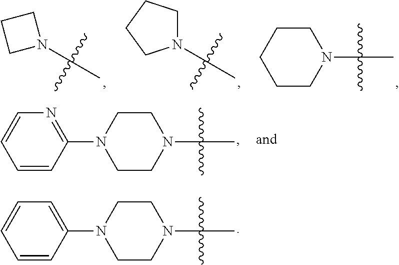

In another embodiment, the present invention provides a method for treating a cellular proliferative disorder in a subject, characterized by a mutation in the p53 gene, comprising administering to the subject in need thereof an effective amount of a compound of Formula (Ia) or (Ib), wherein said compound is selected from the group consisting of NSC319726, NSC319725, and NSC328784.

In another embodiment, the present invention provides a method for treating a cellular proliferative disorder in a subject, characterized by a mutation in the p53 gene, comprising administering to the subject in need thereof an effective amount of a compound of Formula (II):

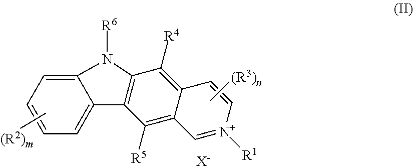

or a pharmaceutically acceptable salt of the compound, wherein:
m and n are each independently 0, 1, 2, or 3;
X⁻ is absent, halide or PtCl₄⁻;
R¹ is absent, H or C₁-C₄ alkyl;
R² at each occurrence selected from C₁-C₄ alkyl, C₁-C₄ alkoxy, R⁷C(O)—, hydroxyl, halogen;
R³, R⁴, and R⁵ are each independently selected from hydrogen, C₁-C₄ alkyl; C₁-C₄ alkoxy, R⁷C(O)—, hydroxyl, and halogen;
R⁶ is hydrogen or C₁-C₄ alkyl; and
R⁷ is C₁-C₄ alkyl.

In another embodiment, the present invention provides a method for treating a cellular proliferative disorder in a subject, characterized by a mutation in the p53 gene, comprising administering to the subject in need thereof an effective amount of a compound of Formula (II), wherein:
m is 1;
n is 0;
X⁻ is iodide or PtCl₄⁻;
R¹ is methyl;
R² is hydrogen, acetyl, hydroxyl or C₁-C₄ alkoxy; and
R⁴, R⁵, and R⁶ are each hydrogen or methyl.

In another embodiment, the present invention provides a method for treating a cellular proliferative disorder in a subject, characterized by a mutation in the p53 gene, comprising administering to the subject in need thereof an effective amount of a compound of Formula (II), wherein said compound is selected from the group consisting of NSC612941, NSC155694, and NSC620256.

In another embodiment, the present invention provides a method for treating a cellular proliferative disorder in a subject, characterized by a mutation in the BRAF gene, comprising administering to the subject in need thereof an effective amount of a compound of Formula (IIIa) or (IIIb):

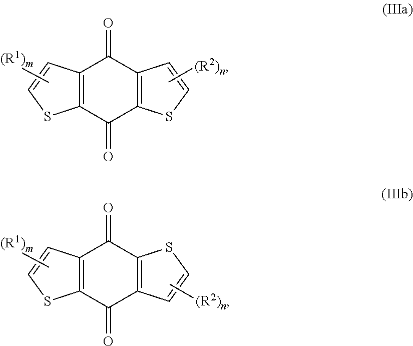

or a pharmaceutically acceptable salt of the compound, wherein:
m and n are independently 0, 1 or 2;
R¹ and R², at each occurrence, are independently selected from C₁-C₆ alkyl, halogen, hydroxyl, C₁-C₆ alkoxy, said alkyl optionally substituted by one to three substituents independently selected from hydroxyl, halogen, and C₁-C₆ alkoxy.

36. The method of claim 35, wherein:
m and n are each independently 0 or 1; and
R¹ and R² are each independently C₁-C₄ alkyl or C₁-C₄ hydroxylalkyl.
In another embodiment, the present invention provides a method for treating a cellular proliferative disorder in a subject, characterized by a mutation in the BRAF gene, comprising administering to the subject in need thereof an effective amount of a compound of Formula (IIIa) or (IIIb), wherein said compound is selected from the group consisting of NSC656238, NSC682449, and NSC690432.

In another embodiment, the present invention provides a method for treating a cellular proliferative disorder in a subject, characterized by a mutation in the KRAS gene, comprising administering to the subject in need thereof an effective amount of a compound of Formula (IV):

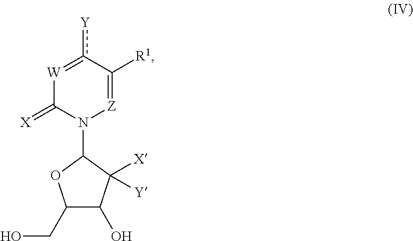

or a pharmaceutically acceptable salt of the compound, wherein:
X is O or S;
Y is NH₂ when C [symbol] Y is a single bond, or O when C [symbol] Y is a double bond;
W is NH when C [symbol] W is a single bond, or N when C [symbol] W is a double bond;
Z is N or CH;
X′ and Y′ are each independently H, hydroxyl or halogen; and
R¹ is H or halogen.

In another embodiment, the present invention provides a method for treating a cellular proliferative disorder in a subject, characterized by a mutation in the KRAS gene, comprising administering to the subject in need thereof an effective amount of a compound of Formula (IV), wherein said compound is selected from the group consisting of NSC613327, NSC146268, and NSC48006.

In another embodiment, the present invention provides a method for treating a cellular proliferative disorder in a subject, characterized by a mutation in the KRAS gene, comprising administering to the subject in need thereof an effective amount of a compound of Formula (V):

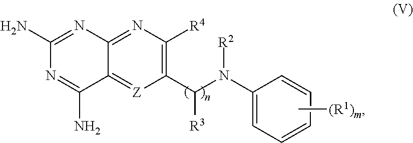

or a pharmaceutically acceptable salt of the compound, wherein:
m is 0, 1, 2, 3, 4, or 5
n is 1, 2, or 3;
Z is N or C—R^{z}, wherein R^{z} is H or C₁-C₄ alkyl;
R¹, at each occurrence, is independently selected from halogen, hydroxyl, C₁-C₄ alkoxy, and R⁵—C(O)—;
R² is hydrogen or C₁-C₄ alkyl;
R³, at each occurrence, is independently hydrogen, C₁-C₄ alkyl, halogen or hydroxyl;
R⁴ is hydrogen, halogen, hydroxyl, or NH₂;
R⁵ is hydrogen, C₁-C₆ alkyl, or —NR^{a}R^{b};
R^{a} and R^{b} are each independently selected from hydrogen, benzyl, and C₁-C₆ alkyl optionally substituted by one, two, or three substituents independently selected from halogen, hydroxyl, —CO₂R⁶, and —SO₃R⁶; and
R⁶ is hydrogen or C₁-C₄ alkyl.

In another embodiment, the present invention provides a method for treating a cellular proliferative disorder in a subject, characterized by a mutation in the KRAS gene, comprising administering to the subject in need thereof an effective amount of a compound of Formula (V), wherein:
m is 1, 2 or 3;
n is 1;
Z is N or C—CH₃;
R¹ is halogen or R⁵—C(O)—;
R² is hydrogen or methyl;
R³ is hydrogen;
R⁴ is hydrogen or NH₂;
R⁵ is hydrogen, C₁-C₆ alkyl, or —NHR^{a};
R^{a} is C₁-C₆ alkyl optionally substituted by one, or two substituents independently selected from —CO₂R⁶, and —SO₃R⁶; and
R⁶ is hydrogen or C₁-C₄ alkyl.

In another embodiment, the present invention provides a method for treating a cellular proliferative disorder in a subject, characterized by a mutation in the KRAS gene, comprising administering to the subject in need thereof an effective amount of a compound of Formula (V), wherein said compound is selected from the group consisting of NSC740, NSC696558, NSC666787, NSC682306, NSC117356, and NSC739.

In another embodiment, the present invention provides a method for treating a cellular proliferative disorder in a subject, characterized by a mutation in the PTEN gene, comprising administering to the subject in need thereof an effective amount of a compound of Formula (VI):

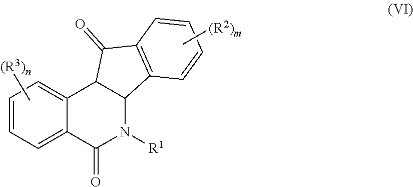

or a pharmaceutically acceptable salt of the compound, wherein:
m and n are each independently 0, 1, 2, 3, or 4.
R¹ is selected from hydrogen, C₁-C₆ alkyl optionally substituted by one, two or three substituents independently selected from the group consisting of hydroxyl, halogen, aryl, heteroaryl, heterocyclyl, and —NR^{a}R^{b};
R² and R³, at each occurrence, are independently selected from hydrogen, halogen, hydroxyl, C₁-C₄ alkoxy, C₁-C₄ haloalkoxy, nitro, aryl, and heterocyclyl, or alternatively, two R² or two R³ combined form a five- or six-membered heterocyclyl ring fused onto the respective benzene ring of the molecule; and
R^{a} and R^{b} are each independently selected from hydrogen, C₁-C₄ alkyl optionally substituted by one or two substituents independently selected from halogen, hydroxyl, and C₁-C₄ alkoxy.

In another embodiment, the present invention provides a method for treating a cellular proliferative disorder in a subject, characterized by a mutation in the PTEN gene, comprising administering to the subject in need thereof an effective amount of a compound of Formula (VI), wherein:
m is 1 or 2;
n is 1 or 2;
R¹ is (CH₂)ᵢ—R^{x}, wherein i is 2, 3, or 4, and R′ is halogen, hydroxyl, heterocyclyl, or substituted alkylamino;
R² is hydrogen, halogen, C₁-C₄ alkoxy, or alternatively, two of R²'s together form a five member-heterocyclyl ring fused onto the benzene ring to which R² is attached; and
R³ at each occurrence is independently selected from C₁-C₄ alkoxy and nitro.

In another embodiment, the present invention provides a method for treating a cellular proliferative disorder in a subject, characterized by a mutation in the PTEN gene, comprising administering to the subject in need thereof an effective amount of a compound of Formula (VI), wherein said compound is selected from the group consisting of NSC706744, NSC735493, and NSC734294.

In another embodiment, the present invention provides a method for treating a cellular proliferative disorder in a subject, characterized by a mutation in the PTEN gene, comprising administering to the subject in need thereof an effective amount of a compound of Formula (VII):

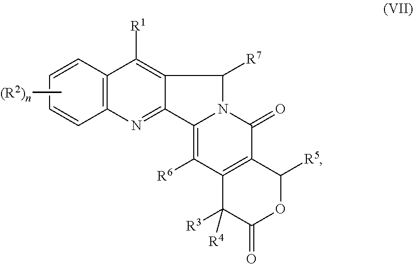

or a pharmaceutically acceptable salt of the compound, wherein:
n is 0, 1, 2, 3, or 4;
R¹ is selected from hydrogen, C₁-C₆ alkyl, hydroxyl, C₁-C₆ alkoxy, halogen, and a group characterized by formula (A):

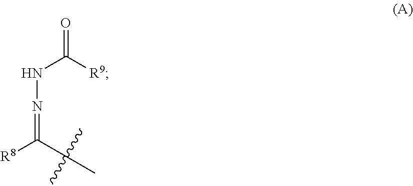

R² at each occurrence is independently selected from hydrogen, C₁-C₆ alkyl, hydroxyl, C₁-C₆ alkoxy, halogen, and —NR^{a}R^{b};
R³ and R⁴ are each independently hydrogen, C₁-C₆ alkyl, hydroxyl, C₁-C₆ alkoxy, R¹²C(O)O—, halogen, and —NR^{a}R^{b};
R⁵ is hydrogen or C₁-C₄ alkyl;
R⁶ is hydrogen, C₁-C₆ alkyl, hydroxyl, C₁-C₆ alkoxy, or halogen;
R⁷ is hydrogen or C₁-C₄ alkyl;
R^{a} and R^{b} are independently hydrogen, C₁-C₄ alkyl, and R¹⁰C(O)—;
R⁸ is hydrogen or C₁-C₄ alkyl;
R⁹ is selected from C₁-C₆ alkyl, aryl, heteroaryl, heterocyclyl, —C(O)NR^{c}R^{d}, wherein said alkyl is optionally substituted by one or two substituents independently selected from the group consisting of hydroxyl, halogen, C₁-C₄ alkoxy, —SR¹¹, aryl, heteroaryl, heterocyclyl, and —NR^{c}R^{d};
R¹⁰ is C₁-C₄ alkyl or NR^{c}R^{d};
R¹¹ is hydrogen or C₁-C₄ alkyl;
R¹² is C₁-C₆ alkyl optionally substituted by NR^{c}R^{d}; and
R^{c} and R^{d} are each independently hydrogen or C₁-C₆ alkyl.

In another embodiment, the present invention provides a method for treating a cellular proliferative disorder in a subject, characterized by a mutation in the PTEN gene, comprising administering to the subject in need thereof an effective amount of a compound of Formula (VII), wherein:
n is 0 or 1;
R¹ is hydrogen or a group of formula (A):

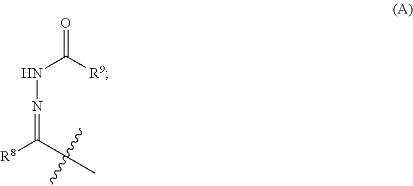

R² is —NHR^{a};
R³ is C₁-C₆ alkyl;
R⁴ is hydroxyl or R¹²C(O)O—;
R⁵ is hydrogen;
R⁶ is hydrogen;
R⁷ is hydrogen;
R⁸ is hydrogen;
R⁹ is selected from heterocyclyl, —C(O)NH₂, and C₁-C₆ alkyl optionally substituted by one or two substituents independently selected from the group consisting of hydroxyl, halogen, C₁-C₄ alkoxy, —SR¹¹, aryl, heteroaryl, heterocyclyl, and NH₂;
R^{a} is hydrogen or R¹⁰C(O)—;
R¹⁰ at each occurrence is independently C₁-C₄ alkyl or NH₂;
R¹¹ (is hydrogen or C₁-C₄ alkyl; and
R¹² is NHR^{c}.

In another embodiment, the present invention provides a method for treating a cellular proliferative disorder in a subject, characterized by a mutation in the PTEN gene, comprising administering to the subject in need thereof an effective amount of a compound of Formula (VII), wherein said compound is selected from the group consisting of NSC681640, NSC681645, NSC681634, NSC681638, NSC606499, NSC606498, NSC606497, NSC364830, and NSC639174.

In another embodiment, the present invention provides a method for treating a cellular proliferative disorder in a subject, characterized by a mutation in the PTEN gene, comprising administering to the subject in need thereof an effective amount of a compound of Formula (VIII):

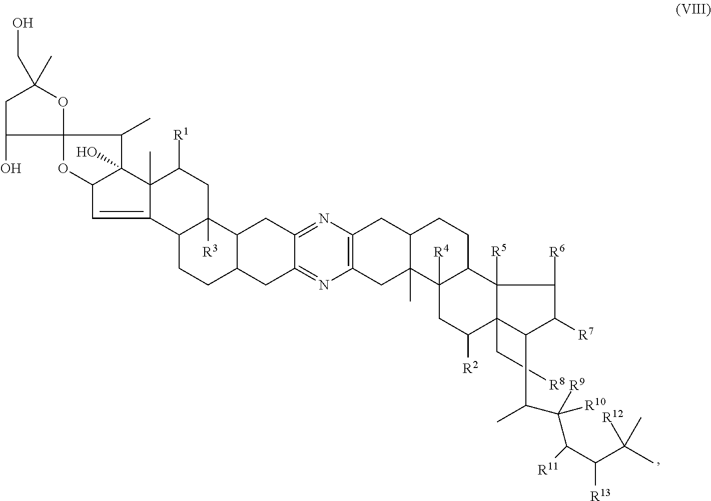

or a pharmaceutically acceptable salt of the compound, wherein:
R¹ and R² are each independently OH or oxo (═O);
R³ and R⁴ are each independently H or OH;
R⁵ and R⁶ together form a bond or an epoxide (—O—);
R⁷ is H or OH, or alternatively in combination with R⁹ or R¹⁰ forms an oxygen bridge (—O—);
R⁸ is OH or alternatively in combination with R⁹ or R¹⁰ forms an oxygen bridge (—O—);
R⁹ and R¹⁰ are each OH, or alternatively in combination with R⁷, R⁸ or R¹² form an oxygen bridge (—O—);
R¹¹ is H or OH;
R¹² is OH, or alternatively in combination with R⁹ or R¹⁰ forms an oxygen bridge (—O—); and
R¹³ is H or CH₃.

In another embodiment, the present invention provides a method for treating a cellular proliferative disorder in a subject, characterized by a mutation in the PTEN gene, comprising administering to the subject in need thereof an effective amount of a compound of Formula (VIII), wherein said compound is one of cephalostatins.

In another embodiment, the present invention provides a method for treating a cellular proliferative disorder in a subject, characterized by a mutation in the PTEN gene, comprising administering to the subject in need thereof an effective amount of a compound of Formula (VIII), wherein said compound is selected from the group consisting of cephalostatin 1 (NSC363979), cephalostatin 2 (NSC363980), cephalostatin 3 (NSC363981), cephalostatin 4 (NSC378727), cephalostatin 8 (NSC378734), and cephalostatin 9 (NSC378735).

It is understood that when a chiral center exists in any of the above structures, the chiral center can take either an R- or an S-configuration. Thus, the structures would encompass all possible stereoisomers, including but not limited to enantiomers and diastereomers.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model. A subject to be treated for a cellular proliferative disorder can be identified by standard diagnosing techniques for the disorder.

Optionally, the subject can then be examined for mutation (e.g., one or more of the mutations discussed herein), expression level, or activity level of an oncogene or a tumor suppressor gene (e.g., the BRAF gene, the p53 gene, the PTEN gene, or the RAS gene) or polypeptide by methods known in the art or described above. If the subject has a particular mutation in the gene, or if the gene expression or activity level is, for example, lower in a sample from the subject than that in a sample from a normal person, the subject is a candidate for treatment.

“Treating” or “treatment” refers to administration of a compound or agent to a subject, who has a disorder (such as a cellular proliferative disorder), with the purpose to cure, alleviate, relieve, remedy, delay the onset of, prevent, or ameliorate the disorder, the symptom of the disorder, the disease state secondary to the disorder, or the predisposition toward the disorder.

An “effective amount” or “therapeutically effective amount” refers to an amount of the compound that is capable of producing a medically desirable result, e.g., as described above, in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The agent can be administered in vivo or ex vivo, alone or co-administered in conjunction with other drugs or therapy. As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, a compound is administered to a subject. Generally, the compound is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

In the following examples, the applicability of the methodology disclosed herein was tested and corroborated. The input data was obtained from the National Cancer Institute anticancer drug screen, NCI60 screen (October 2009 release, which is available at dtp.nci.nih.gov/docs/cancer/cancer_data.html).

### Example 1

### Compounds with Increased Activity in Cells Carrying BRAF^{V600E} Mutation

Oncogenenic mutations in the BRAF gene correlate with increased severity and decreased response to chemotherapy in a wide variety of tumors. The BRAF gene encodes a protein kinase (B-Raf) and mutations in this gene can result in a constitutive activation of its kinase, promoting cell proliferation. Among these oncogenic mutations, BRAF^{V600E} is one of the most prevalent mutations in tumors and the most frequent oncogenic protein kinase mutations known. A potent inhibitor of the B-Raf activity (AZD6244) has been recently reported (Yeh, T. C., et al., Clin Cancer Res, 2007. 13(5): p. 1576-83). AZD6244 manifests selective growth inhibition of tumor derived cell lines with mutations in B-Raf and members of the Ras receptor kinases. This compound is in Phase II clinical trials in cancers with BRAF mutations (NCT00888134) and was tested within the NCI60 screen, offering a unique opportunity to test the applicability of the methodology described above.

Within the cell lines in the NCI60 screen, 11 lines carry a BRAF^{V600E} mutation (Ikediobi, O. N., et al., Mol Cancer Ther, 2006. 5(11): p. 2606-12). Furthermore, 10 of those also carry a mutation in TP53, CDKN2A or both. The TP53 gene encodes the transcription factor p53 that plays a central role in tumor suppression. In turn, ARF, one of the gene products of the CDKN2A gene, is a positive regulator of p53. Inactivating alterations of these two genes promote tumor formation and correlate with decreased response to chemotherapy. This correlation, between carrying a BRAF^{V600E} mutation and a mutation in TP53 or CDKN2A, should be taken into account when constructing the control group of cell lines with wild-type BRAF. Thus, as a control group, the 10 NCI60 cell lines with wild-type BRAF but carrying a mutation in either TP53 or CDKN2A were selected. The resulting groups are the following:

### Case Group:

Melanoma: LOXIMVI, MALME-3M, SK-MEL-2, SK-MEL-5, SK-MEL-28, M14, UACC-62, UACC-257, MDA-MB-435;
Colorectal Cancer: HT29, COLO-205.

### Control Group:

Breast Cancer: HS578T, MCF7;
Central Nervous System Cancer: U251;
Colorectal Cancer: KM12;
Lung Cancer: EKVX;
Leukemia: SR;
Ovarian Cancer: IGROV1;
Renal Cancer: A498, ACHN, SN12C.

Using the protocols described above, the NCI60 treatment-cell line RG was constructed, taking RS=log₁₀IC50-average(log₁₀IC50) as a relative response sensitivity measurement and computing the average(log₁₀IC50) over all the 60 cell lines. The Case and Control groups listed above were chosen, and the score using Formula I was computed for the 47,624 compounds in the NCI60 screen, including AZD6244 under the NCI designation NSC741078.

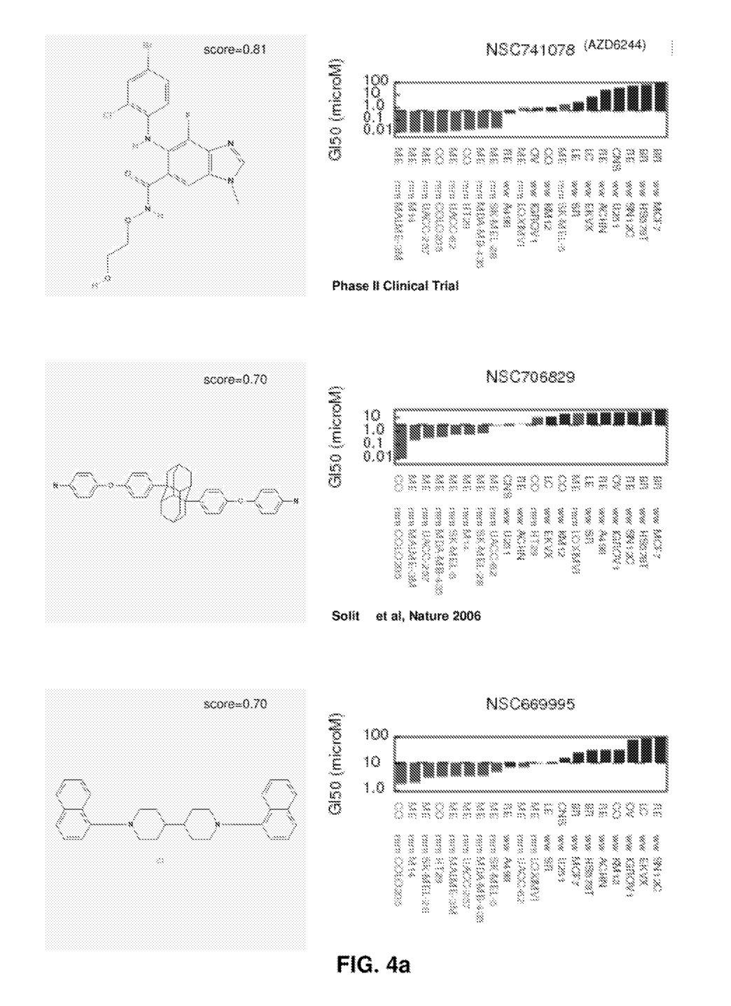

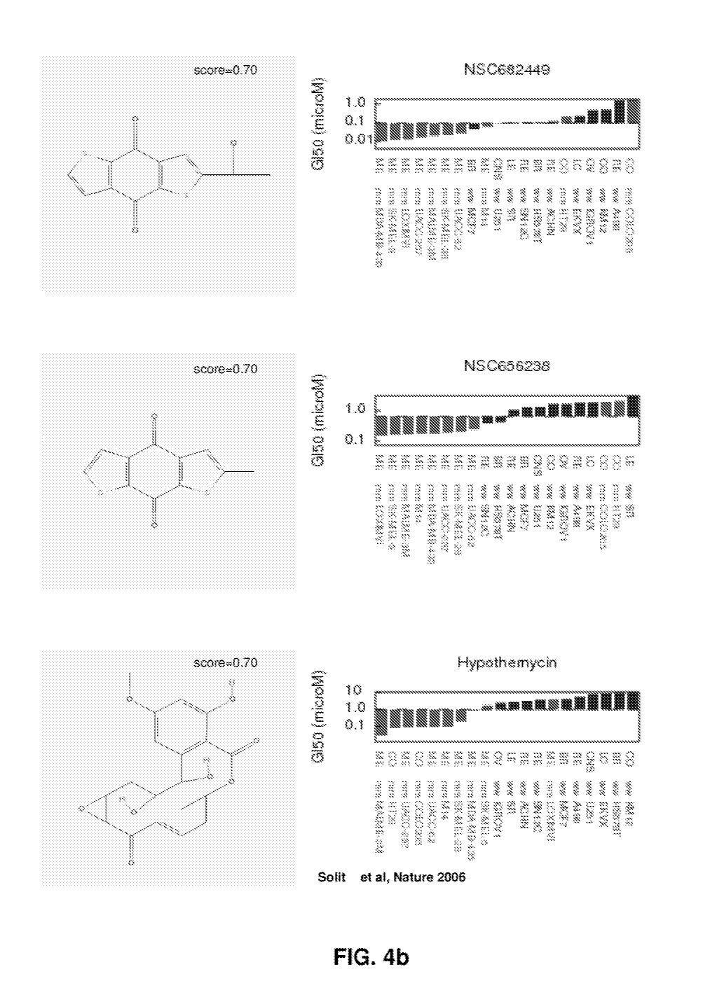

Six compounds with highest score were identified and shown in FIGS. 4A and 4B, where the left panels show the compounds' chemical structures, while the right panels show their pattern of growth inhibition across the cases and controls, where GI50 is the IC50 measure reported by the NCI60 screen. Among the six compounds, it was found that AZD6244 had the highest score, thereby proving principles for the method disclosed in this application. Two other compounds, NSC706829 and Hypothemycin, have been already identified in a previous screen using a different methodology (Solit, D. B., et al., Nature, 2006. 439(7074): p. 358-62). Thus, these results demonstrate that the methodology disclosed in this application can be used to identify compounds for treating cancer with BRAF^{V600E} mutation. For the same reasons, the three other compounds, NSC669995, NSC682449 and NSC656238, represent novel chemotherapeutic agents with selective anticancer inhibition of BRAF^{V600E} tumors.

### Example 2

### Compounds with Increased Activity in Cells Carrying p53 Mutations

Mutations in the TP53 gene correlate with increased severity and decreased response to chemotherapy in a wide variety of tumors. The TP53 gene encodes a transcription factor that plays a central role in tumor suppression and mutations in this gene can result in lost of the p53 tumor suppressor function and gain of function as well. TP53 mutations are the most frequent mutations in tumors. Most occurring mutations are observed within the p53 DNA binding domain, with hot spots at amino acids 273 and 248 in the p53 protein, right at the point of contact with DNA, and at amino acid 175, in the zinc pocket region.

Among the NCI60 cell lines, 16 have wild-type p53 and 44 carry a p53 alteration (Ikediobi, O. N., et al., Mol Cancer Ther, 2006. 5(11): p. 2606-12). Mutations at amino acids 273 and 248 are the most frequent, with 5 and 4 cell lines, respectively. Given the abundance of p53 mutations at positions 273 and 248 within the NCI60 cell lines, and their location in the p53-DNA contact, cells carrying these mutations were chosen to form a case group to identify compounds with increased activity in tumors with a mutant p53. On the other hand, cells with a wild-type p53 were chosen to form a control group. The resulting groups are the following:

### Case Group:

Leukemia: CCRF-CEM;
Lung Cancer: NCI-H322M;
Central Nervous System Cancer: SF-295, SF-268, SNB-19, and U251;
Colorectal Cancer: HT29 and SW-620;
Ovarian Cancer: OVCAR-3.

### Control Group:

Breast Cancer: MCF7;
Colorectal Cancer: HCT-116;
Lung Cancer: A549, NCI-H226, and NCI-H460;
Leukemia: SR;
Melanoma: LOXIMVI, MALME-3M, SK-MEL-5, UACC-257, and UACC-62;
Ovarian Cancer: OVCAR-5;
Renal Cancer: A498, ACHN, CAKI-1, and UO-31.

Using the protocols described above, the NCI60 treatment-cell line RG was constructed, taking RS=log₁₀IC50-average(log₁₀IC50) as a relative response sensitivity measurement and computing the average(log₁₀IC50) over all the 60 cell lines. The Case and Control groups listed above were chosen, and the score in Formula I was computed for the 47,624 compounds in the NCI60 screen. The six compounds ranking highest were identified as NSC612941 (0.55), NSC155694 (0.55), NSC319726 (0.52), NSC319725 (0.50), NSC694266 (0.50) and NSC93739 (0.50), their scores being indicated within the parenthesis.

None of these compounds have been previously reported for their selective activity in cells with a mutant p53. To provide further evidence for their specificity, growth inhibition assays were performed on isogenic cell lines. Previously developed murine fibroblast cell line (10)3 (p53 null) and 10(3) with transfection of cytomegalovirus-human mutant p53 constructs (273, 248 and 175) (Dittmer, D., et al., Nature Genetics, 1993. 4(1): p. 42-6) were used as cases, while the murine fibroblast cell line 3T3 was used as a p53 wild type control. The six compounds ranking highest were requested from the NCI and three of them (NSC155694, NSC319725 and NSC319726) were obtained.

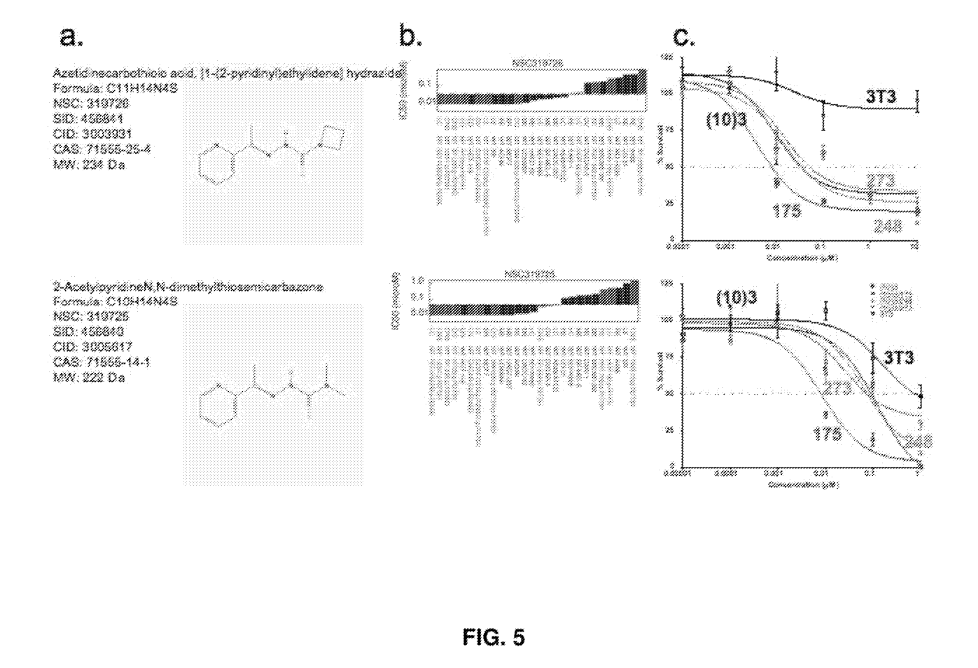

It was found that Compounds NSC319725 and NSC319726 exhibited a selective activity as predicted, with a higher sensitivity in cells with a p53 alteration (FIG. 5). However, the growth inhibition curves manifested clear allele specific effects. Cells carrying the p53¹⁷³ mutation showed IC50s in the 0.01 micromolar range; those with mutations p53²⁷³, p53²⁴⁸ and p53^{null} in the 0.1 micromolar range. In contrast, p53 wild type cells manifested the highest IC50s, which were above 1 micromolar. These results corroborate the selective activity of the compounds identified using the method described in this application. The cell lines with a p53 mutation or absence of p53 exhibit IC50s at least a 10 fold lower compared to that of the p53 wild-type control. This evidence further demonstrates that the method described in this application can be used to identify compounds for treating cancers, in particular, cancers with a specific gene mutation.

### Example 3

### Allele Specific p53 Mutant Synthetic Lethality

In this example, NSC319725 and NSC319726 were found to be synthetic lethal for cell lines expressing mutant p53. These two compounds belong to the same family—the thiosemicarbazones. The chemical structures for these compounds are shown in FIG. 5a along with plot of the IC50's in a sample of the 60 tumor cell lines that make up the NCI60. The clustering of the most sensitive cell lines is made up mostly of tumor cell lines containing mutant p53. See FIG. 5b, showing the clustering of the cell lines with the sensitivity to the two compounds, based on computational analysis of the NCI60 database. Red bars represent the cell lines with the three hotspot mutations of p53 (R175, R248 and R273). Blue bars represent the cell lines with wild type p53.

The compounds were validated using several different cell line systems. The first system used was a mouse embryonic fibroblast (MEF) system in which a p53 null MEF line, 10(3), was used as a parental line from which several stable CMV-mutant p53 transfectants were derived. The p53 mutations investigated were the “hot spot” mutants 175, 248, and 273. NIH3T3 fibroblasts were used as a p53 wild type control. It was found that both of the compounds exhibited markedly higher sensitivity in cell lines expressing mutant p53 as compared to the wild type control, FIG. 5c. Most notably, the highest sensitivity was observed in cells with the 175 allele. In fact, the IC50 for the 175 mutant was more than 100 fold lower than that of the wild type. As shown in FIG. 5c, for NSC319726, the IC50 for the 175 mutant was 8 nM while the IC50 of the wild type cell line was not even reached.

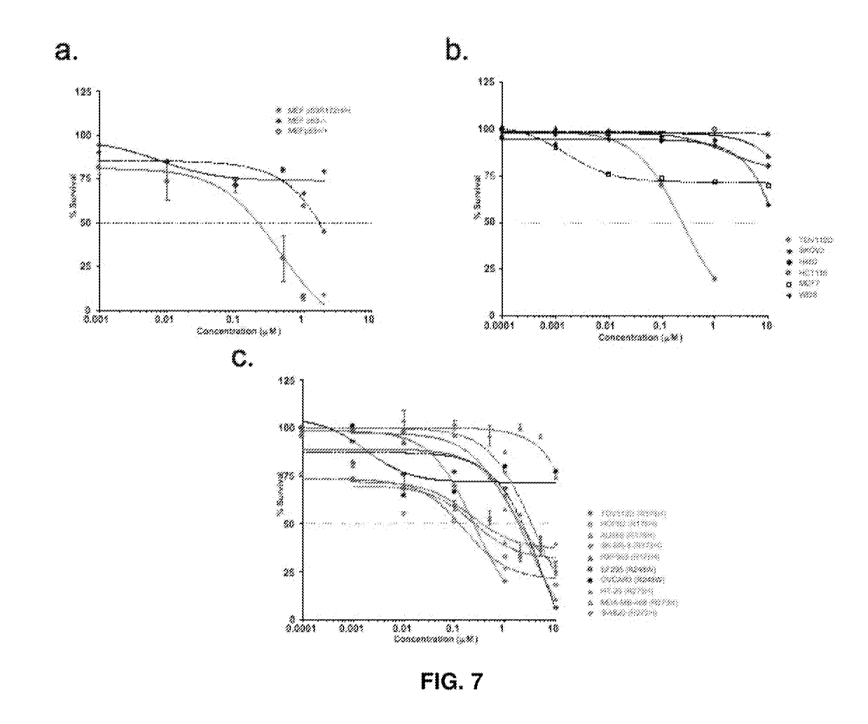

Assays were carried out to further validate NSC319726 in other p53 mutant cell line systems. Using a system of mouse embryonic fibroblast (MEF) cell lines derived from the same strain, the sensitivities of MEF-p53 wild type, MEF-p53 null, and MEF-p53^{R172H} cells to NSC319726 were compared. The sensitivity was measured in 12-well cultures for viability assay using the Guava PCA instrument following the manufacture protocol. It was found that again NSC319726 exhibited a much higher sensitivity for the MEF-p53^{R172H} as compared to the p53 wild type and p53 null controls, FIG. 7a.

Next, assays were carried out to examine the sensitivity to NSC319726 of a human tumor cell line system that was made up of 4 cell lines expressing wild type p53, one cell line expressing p53 null cell in (SKOV3), and one ovarian carcinoma cell line containing a p53^{R175H} (TOV112D). It was noted that the TOV112D cell line had a marked sensitivity and the p53 wild type cell lines showed a relative lack of sensitivity at a concentration rage from 0.1 μM to 10 μM of 319726, FIG. 7b. To further demonstrate the 175 allele specificity of NSC319726, assays were performed to compare the sensitivities across human tumor cell lines with different p53 “hot spot” mutations. Five tumor cell lines with endogenous R175H mutations were compared, including two cell lines containing R248W mutations and three cell lines with a R273H mutation. Once again, with the exception of one R175H cell line (RXF393), the R175H cell lines exhibited similar sensitivities that were approximately 10-fold and in some instances 100-fold higher than the other p53 mutant alleles, FIG. 7c.

### Example 4

### Mechanism of NSC319726's Mutant Synthetic Lethality

In this example, assays were performed to demonstrate that the mechanism of NSC319726's mutant synthetic lethality is by induction of apoptosis that is p53^{R175H} dependent.

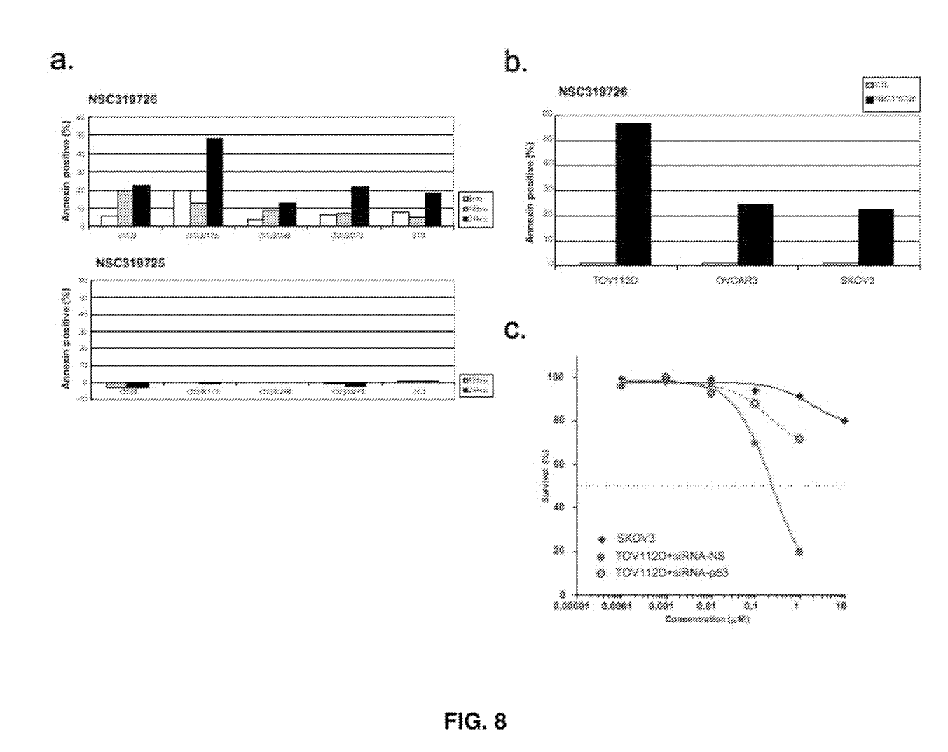

Briefly, apoptosis was examined by Annexin V staining using standard methods. The Annexin V staining of NSC319726 treated MEF cells indicated that the mechanism of growth inhibition was by induction of apoptosis. As shown in FIG. 8a, NSC319726 induced apoptosis in MEF cells with exogenous p53 mutations and (10)3/175 displayed strongest apoptosis response to the compound. In contrast, NSC319725 did not induce apoptosis, suggesting that, interestingly, the mechanism of NSC319725 does not seem to involve apoptosis, FIG. 8a.

In addition, treatment of three ovarian carcinoma cell lines (TOV112D-p53R175H, OVCAR3-p53 R248W and SKOV3-p53 null) revealed an induction of apoptosis in all three that was maximally seen in the TOV112D cell line. As shown in FIG. 8a, NSC319726 induced apoptosis in TOV112D, which show almost 3-fold more Annexin V staining than the other two ovarian cell lines.

Because NSC319726 displayed such a marked sensitivity for cell lines expressing the R175H p53 mutant allele, it was hypothesized that the mechanism involved the p53^{R175H} mutant protein. In fact, when expression of the p53^{R175H} mutant protein was silenced in the TOV112D cells by siRNA, it was observed that the sensitivity of TOV112D was reduced by four-fold, FIG. 8c. In contrast, the sensitivity of the p53 null cell line (SKOV3) was not reduced by the siRNA in a similar fashion. This result demonstrated that the mechanism of NSC319726 is dependent on the p53^{R175H} mutant protein.

### Example 5

### NSC319726 Induced “Wild type-Like” Conformational Change in p53^{R175H} Mutant Protein via its Azetidine Ring Side Group

As apoptosis induced by NSC319726 was dependent on the p53^{R175H} mutant protein, assays were carried out to determine if this compound induced a conformational change in the mutant protein structure.

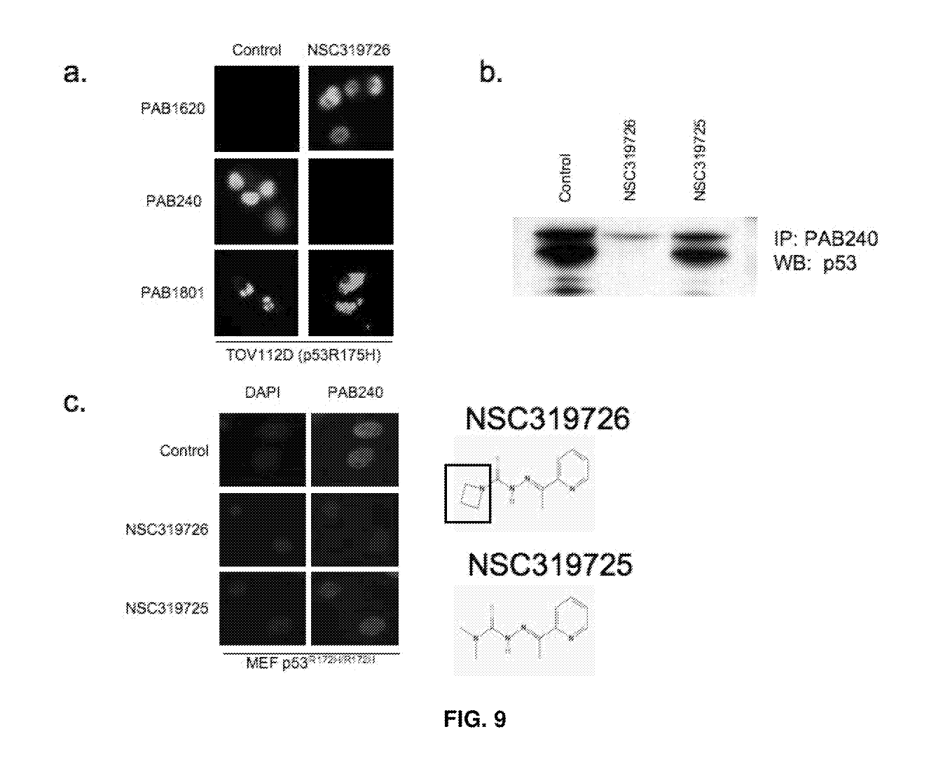

Conformational change to wild type in TOV112D (p53R175H) was studied by immuno-fluorescent staining. Briefly, TOV112D cells were grown on coverslips, treated with NSC319726 and stained with conformation-specific antibodies PAB1620, which only recognizes wild type p53 conformation, PAB240, which only recognizes mutant p53 conformation, and PAB1801, which can recognize both. As shown in FIG. 9a, it was observed that indeed NSC319726 induced a change in conformation of the p53^{R175H} mutant protein to a structure that could be recognized by the p53 wild type specific antibody (PAB1620) but was no longer recognized by the p53 mutant specific antibody (PAB240).

This conformational change was confirmed by immunoprecipitation of lysates from NSC319726-treated TOV112D cells using the mutant specific antibody PAB240. To that end, cell lysates were precipitated with PAB240 and detected with p53 (DO-1) antibody which recognizes all types of p53 protein conformations. It was found that NSC319726 treatment, but not NSC319725, greatly reduced the amount of p53 R175H that can be immunoprecipitated by the mutant specific antibody, FIG. 9b. The fact that this conformation change in the p53^{R175H} mutant protein occurred upon NSC319726 treatment but not with NSC319725 treatment, implicates that the azetidine ring of NSC319726 is the functional side group of this compound, FIG. 9c.

### Example 6

### “Wild type-Like” Conformational Change Induced by NSC319726 Restored Site-Specific p53 Transactivational Function

To determine if the conformation change caused by NSC319726 was functional, Western Blot assays were performed to examine p21 protein levels in TOV112D (p53R175H) and SKOV3 (p53 null) cells treated with NSC319725 and NSC319726.

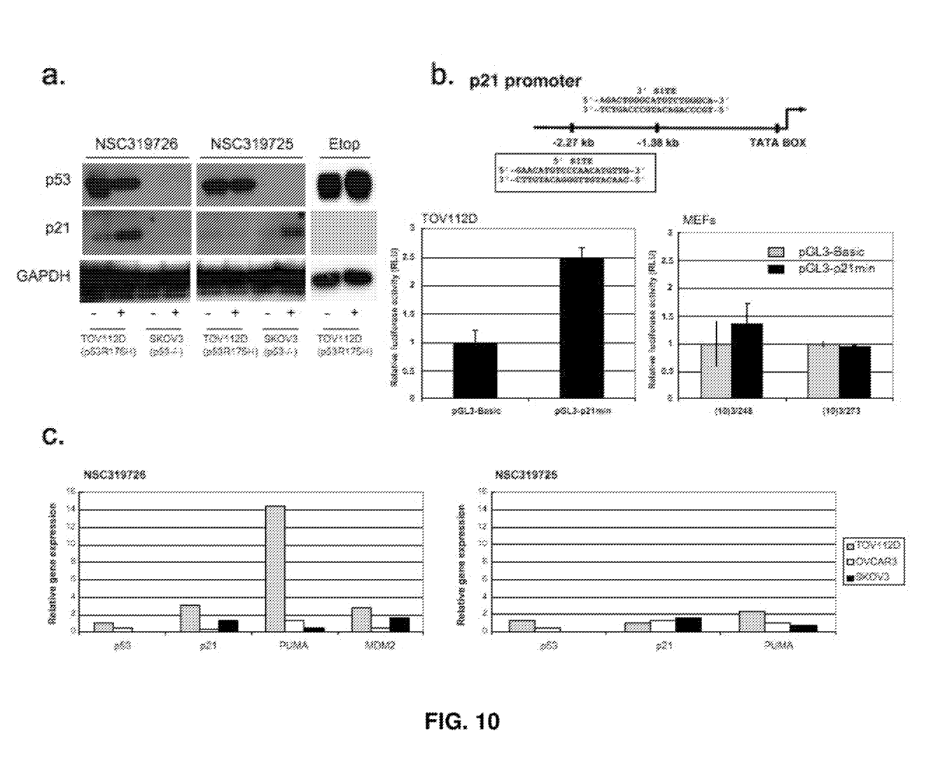

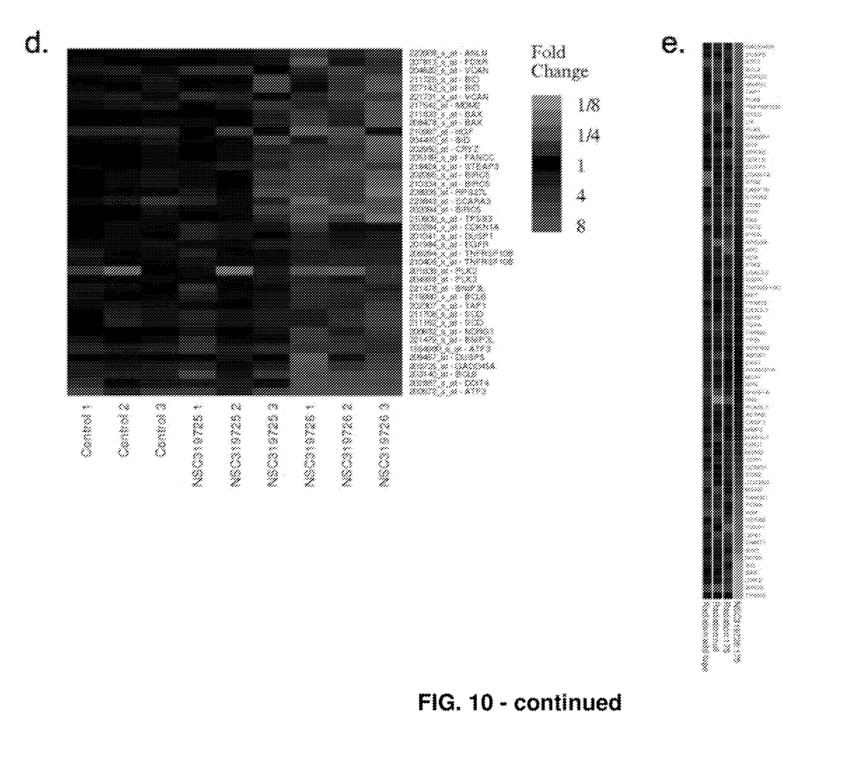

It was found that NSC319726 treatment induced p21 in the TOV112D line but not in the SKOV3 line, FIG. 10a. NSC319725 treatment did not induce p21 in the TOV112D line, but did induce p21 in the SKOV3, demonstrating a p53 independent induction of p21. Treatment of the TOV112D cells with the DNA damaging agent, Etoposide failed to induce p21 suggesting that the p21 induction seen with NSC319726 treatment was in fact p53 dependent, FIG. 10a.

To provide further evidence that NSC319726 restored site-specific p53 transactivational function. To that end, luciferase activity assays were performed. Briefly, a 20-bp p53 response element (p53RE) was subcloned in the pGL3 vector to generate a luciferase reporter plasmid, which contained 20 base pairs of the p53 response element in the p21 promoter. The sequences of the 5′ site and 3′ site at −2.27 kb and −1.38 kb (SEQ ID NOs: 1 and 3) and their reverse complements (SEQ ID NOs: 2 and 4) are listed below:

5′ SITE:
SEQ ID NO: 1
5′ GAACATGTCCCAACATGTTG 3′
SEQ ID NO: 2
5′ CAACATGTTGGGACATGTTC 3′
3′ SITE:
SEQ ID NO: 3
5′ AGACTGGGCATGTCTGGGCA 3′
SEQ ID NO: 4
5′ TGCCCAGACATGCCCAGTCT 3′

The reporter plasmid was transfected to the TOV112D cells or MEF cells with exogenous 248 or 273 mutations. Cells thus-transfected were treated with NSC319726 and subjected to luciferase assays. It was found that, upon NSC319726 treatment, a 2.5 fold increase in luciferase activity in the TOV112D cells was observed. This effect was not seen in MEF cell lines expressing the 248 and 273 alleles, FIG. 10b.

Assays were carried out to compare the mRNA levels of several p53 targets (p21, PUMA, and MDM2) in the TOV112D (p53R175H), OVCAR3 (p53R248W) and SKOV3 (p53 null) upon treatment with the compounds. The relative gene expression level was normalized with the actin and the ratio of the treated level vs. untreated level was shown for each cell line. It was found that NSC319726 induced all three targets in the TOV112D cells, particularly the apoptotic gene PUMA, while no such effect was observed upon NSC319725 treatment, FIG. 10c.

To examine the transcriptional activity of the NSC319725 and NSC19726-treated cells on a larger scale, microarrays were used to analyze control- and compound-treated TOV112D cells. Shown in FIG. 10c, is a heat map that displays the transcriptional levels of a panel of p53 targets relative to the levels detected in three independent untreated TOV112D controls. Most notably, NSC319726 treatment induced a large proportion of these genes while at the same time repressing a large fraction as compared to the untreated controls, providing further evidence that NSC319726 treatment restores p53^{R175H} transactivational activity.

Next, assays were performed to compare the signature of the NSC319726-treated cells to a signature that would be indicative of wild type p53 upon an activating stimulus such radiation. As shown in FIG. 10e, p53 signatures of NSC319726-treated cells were quite different from those of wild type p53 upon activation by gamma radiation. Also displayed in Also shown in FIG. 10e, are the signatures of several p53 null and p53R175H cell lines in response to radiation as controls.

### Example 7

### NSC319726 Demonstrated in-vivo p53^{R175H} Mutant Synthetic Lethality

Toxicity assays were performed in p53 wild type, p53 null, and p53^{R172H} mice to determine if in vivo p53 mutant synthetic lethality could be detected. It was hypothesized that p53^{R172H} mice would experience greater toxicity for a given dose of NSC319726 as compared to the p53 wild type or null mice.

First, three groups of mice: p53^{+/R172H} wild type, p53^{+/R172H/R172H} mice, were compared. NSC319726 was injected intraperitoneally at 10 mg/kg daily for seven days. It was found that by day 3 all seven of the p53^{R172H/R172H} mice had died while only 1 in 9 p53 wild type mice had died. By day 4, the survival of the p53 wild type mice fell to 70% while the p53^{+/R172H} had fallen to 30%, suggesting a clear effect that is dependent on the TP53 status. By day seven approximately 40% of the wild type mice were alive, suggesting that there is some toxicity of the drug at this dose that is not p53 dependent.

Next, the dose was lowered to 5 mg/kg and three groups of mice (p53 wild type, p53 null, and p53^{R172H/R172H}) were compared. At this dose, it was found that, by day seven, the p53 wild type and p53 null mice exhibited a 100% survival compared to only 30% in the p53^{R172H/R172H} mice.

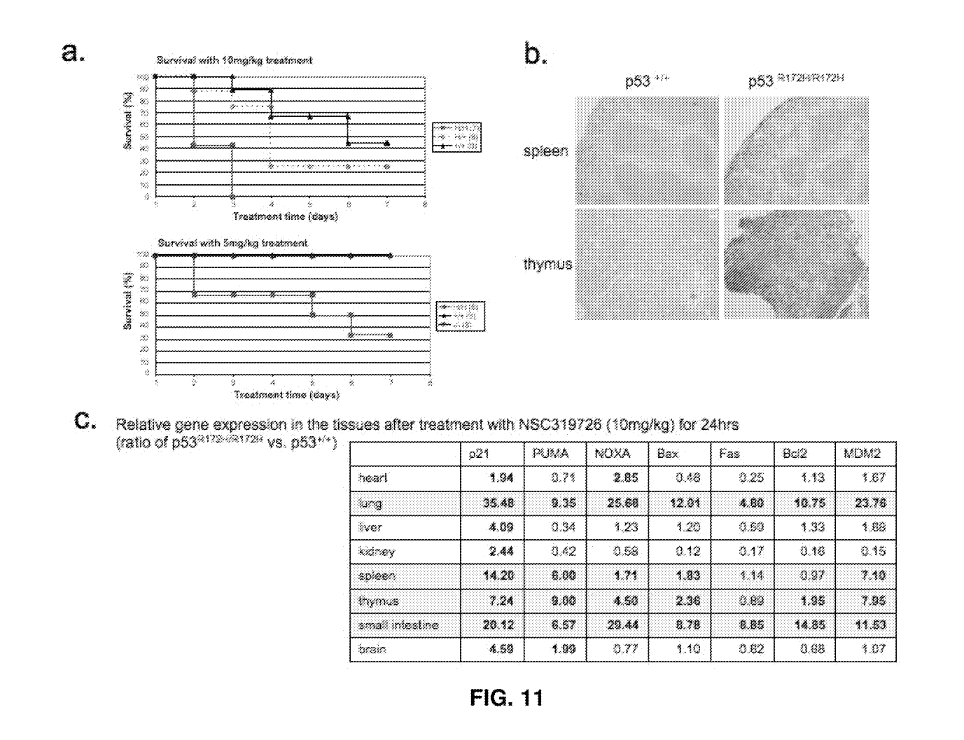

Tissues of p53 wild type and p53^{R172H/R172H} mutant mice after treatment for 24 hours with NSC319726 were examined for evidence of apoptosis by cleaved Caspase-3 immuno-staining as well as gene expression of a panel of p53 targets by quantitative-PCR. As shown in FIG. 11b, abundantly more cleaved Caspase-3 immuno-stained cells were detected in sections of the spleen and thymus of p53^{R172H/R172H} mice as compared with the wild type controls. Elevated mRNA levels of a number of p53 targets were also detected over a range of tissues in the p53^{R172H/R172H} mice as compared with the wild type controls, most notably in the lung, spleen, thymus and small intestine, FIG. 11c. Taken together, these findings provide in-vivo evidence for p53 mutant synthetic lethality by NSC319726 and indicate that NSC319726 can restore the function of p53^{R175H} protein in-vivo.

### Example 8

### NSC319726 Treatment Inhibited Mouse Xenograft Tumor Growth in an Allele Specific Mutant P53 Dependent Manner

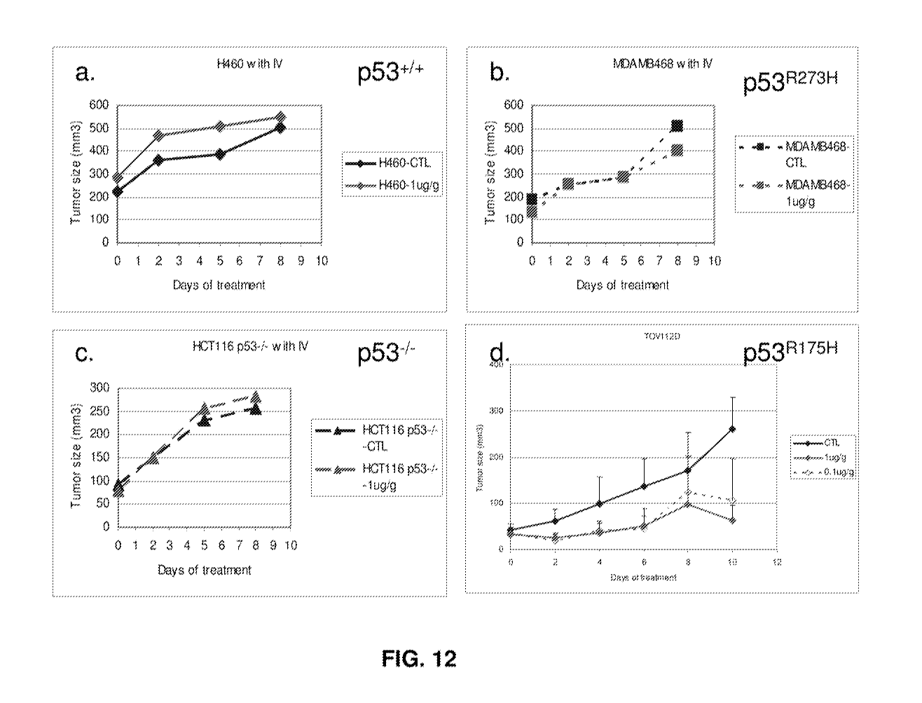

Xenograft tumors were created by subcutaneous flank injections of 8×10⁶ human tumor cells and allowed to grow to an initial size ranging from 50-200 mm³ prior to initiation of NSC319726 (1 μg/g) (versus control, “ctl”) administered intravenously once daily. As shown in FIG. 12, tumor cell lines examined included (a) H460-p53 wildtype (n=1), (b) MDAMB468-p53^{R273H} (n=1), (c) HCT116-p53 null (n=1) and (d) TOV112D-p53^{R175H} (n=7). It was found that NSC319726 inhibited mouse xenograft tumor growth in an allele specific mutant p53 dependent manner. Note there was no difference in growth inhibition when the dose of NSC319726 was lowered by 10-fold (0.1 μg/g), (n=7). See FIG. 12d. These results again demonstrate that NSC319726 exhibited a much higher sensitivity for the tumor with p53^{R172H} mutations.

### Example 9

### Identification of Compounds with Increased Activities Against Cells Having Mutations in Different Genes

The methodology described in this application was used to further screen compounds in the NCI60 screen in the manner described in Examples 1 and 2 above for compounds having increased activity against cells having mutations in four genes: p53, BRAF, KRAS, and PTEN. Listed below are 46 identified compounds:

1. 7 compounds identified as being specific for disorders characterized by a mutation in the p53 gene:

### NSC319726, NSC319725, NSC328784, NSC612941, NSC155694, NSC694266, and NSC93739.

2. 9 compounds identified as being specific for disorders characterized by a V600E mutation in the BRAF gene:

### NSC656238, NSC682449, NSC690432, NSC741078, NSC706829, NSC669995, NSC361127, NSC263637, and NSC354462.

3. 11 compounds identified as being specific for disorders characterized by a mutation in the KRAS gene:

### NSC613327, NSC146268, NSC740, NSC696558, NSC666787, NSC682306, NSC117356, NSC739, NSC680417, NSC363981, and NSC266046.

4.22 compounds identified as being specific for disorders characterized by a mutation in the PTEN gene:
NSC706744, NSC735493, NSC734294, NSC681640, NSC681645, NSC681634, NSC681638, NSC606499, NSC606498, NSC606497, NSC364830, NSC639174, NSC620256, NSC363979, NSC363980, NSC363981, NSC378734, NSC378735, NSC378727, NSC355447, NSC368891, and NSC48006.

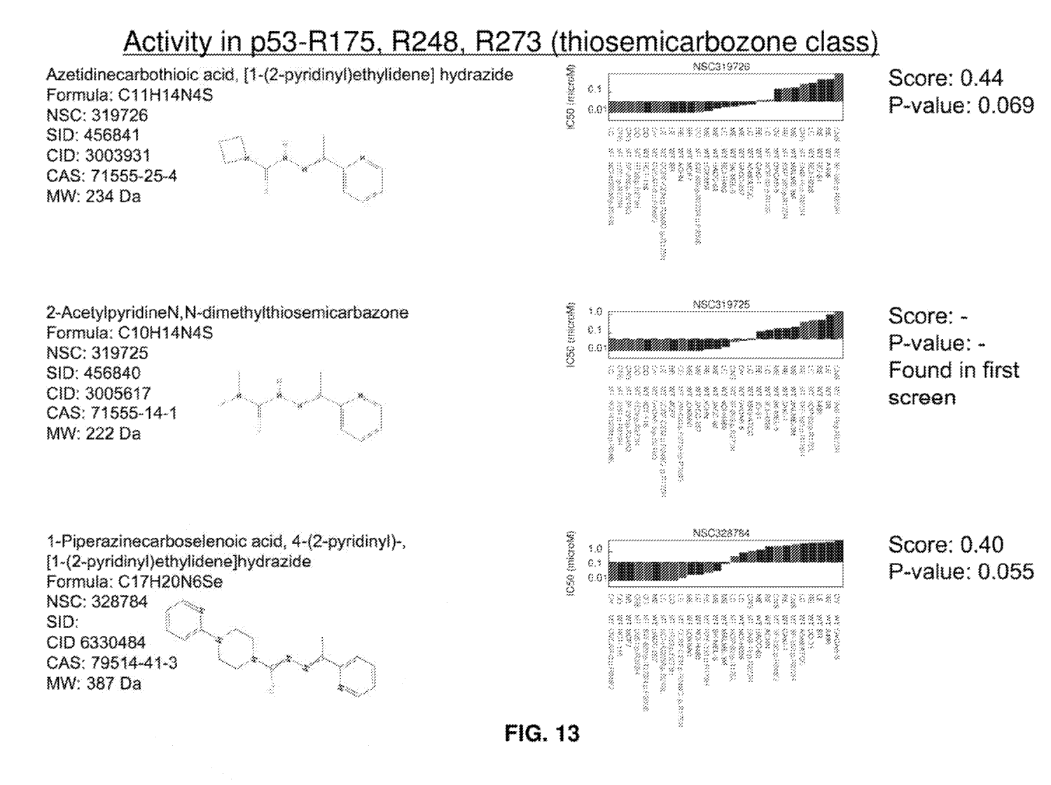

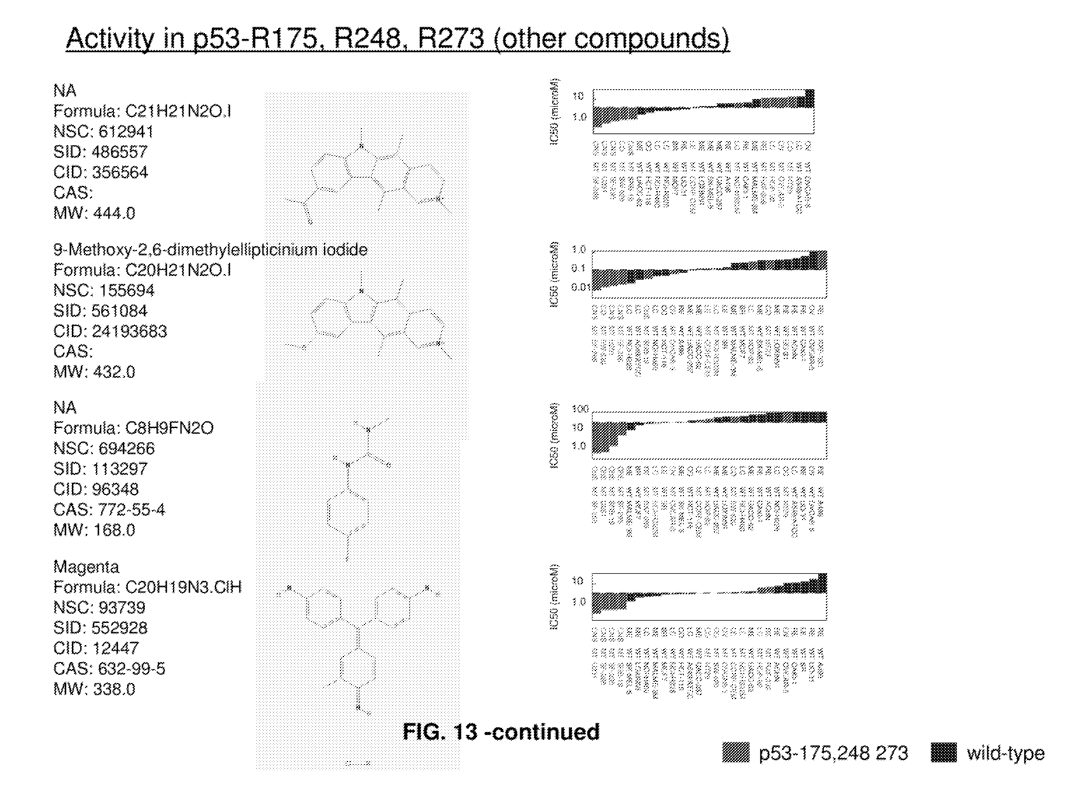

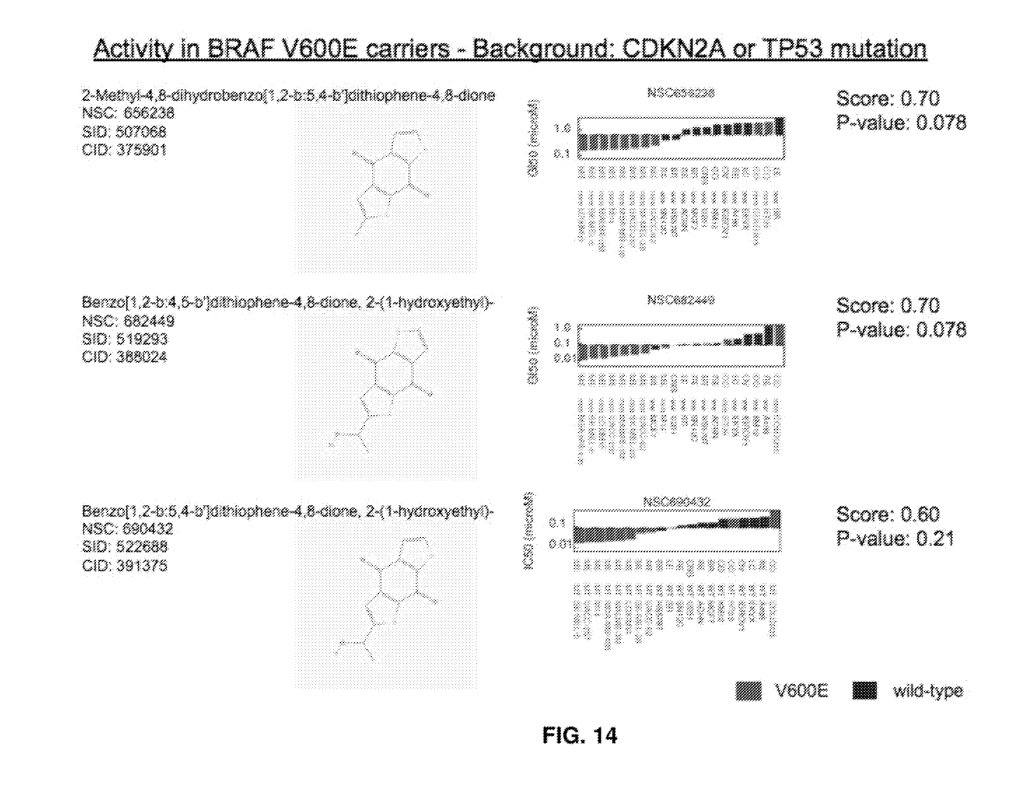

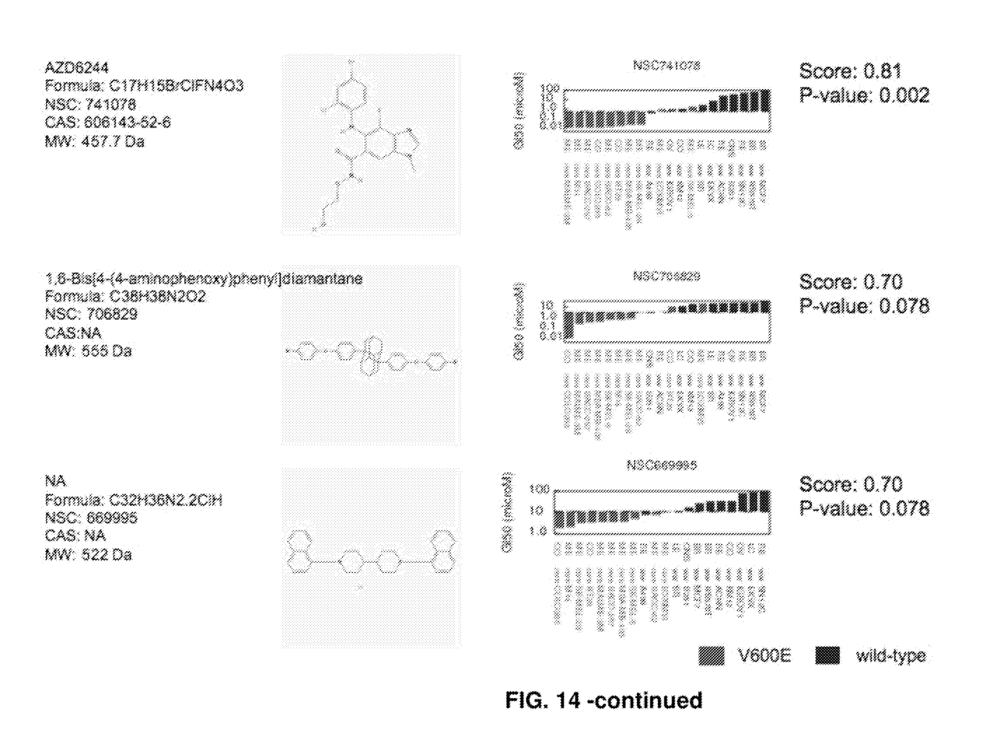

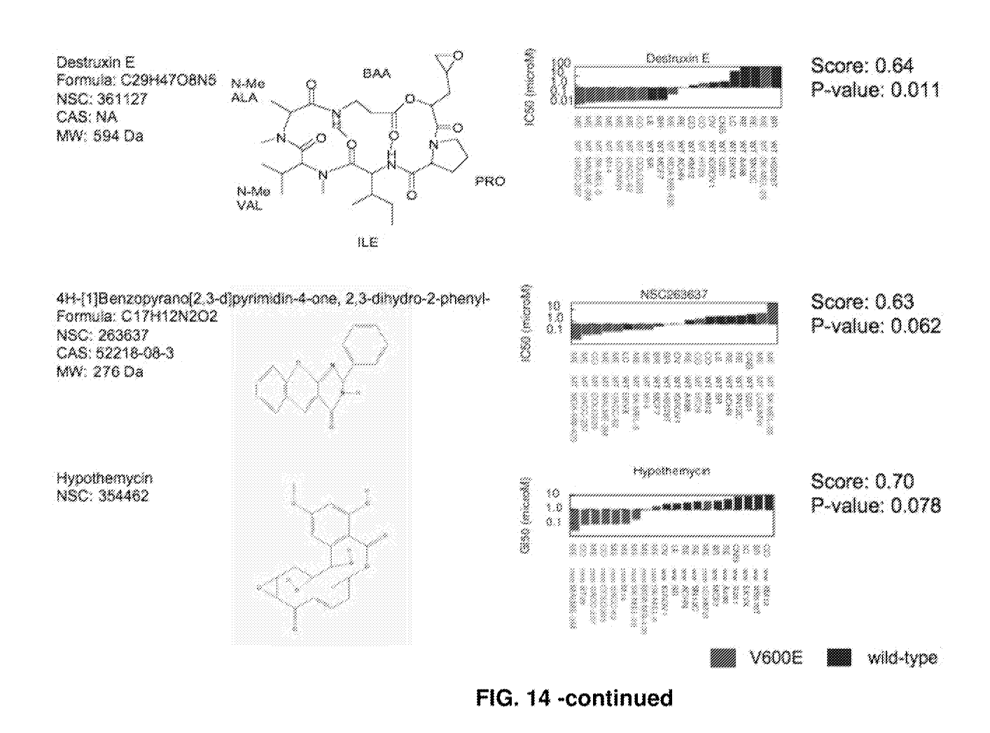

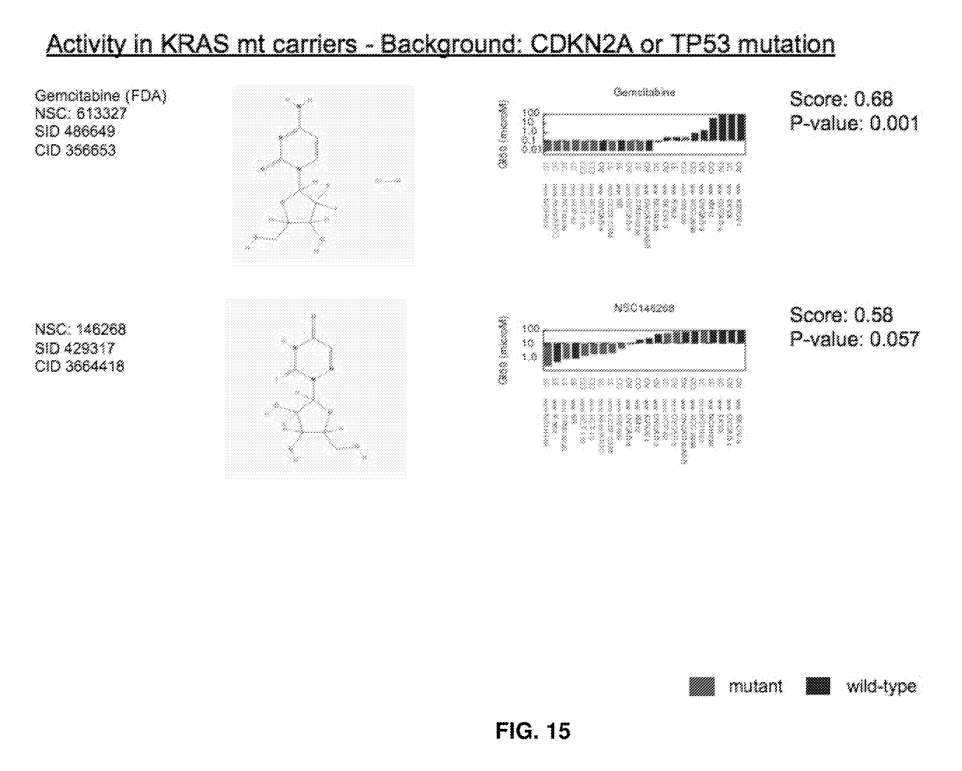

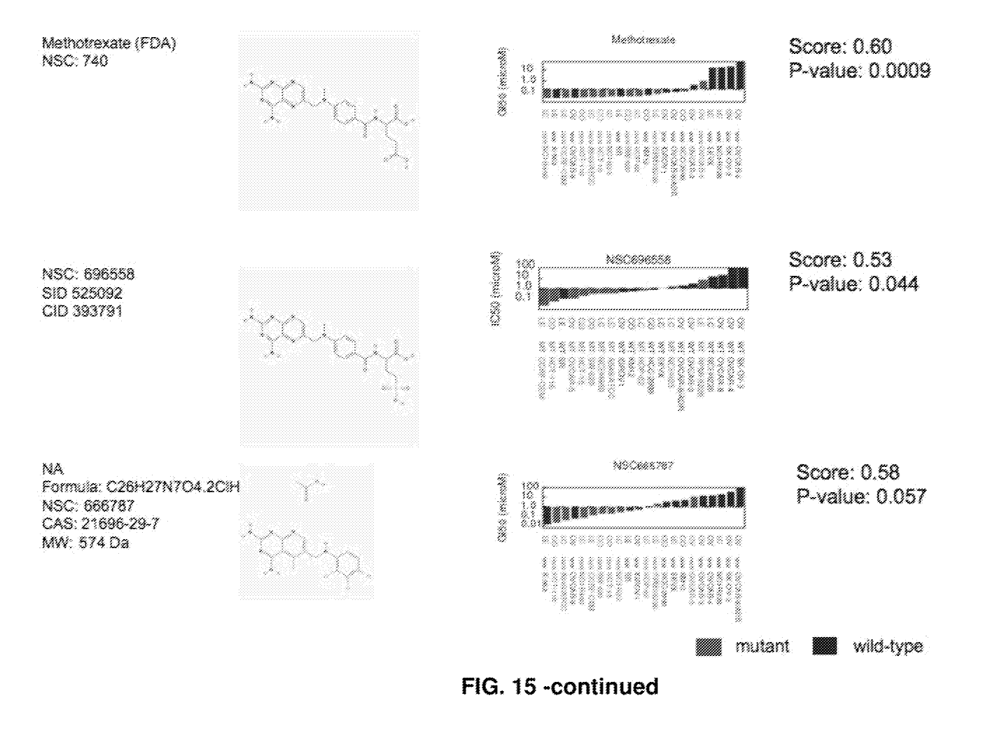

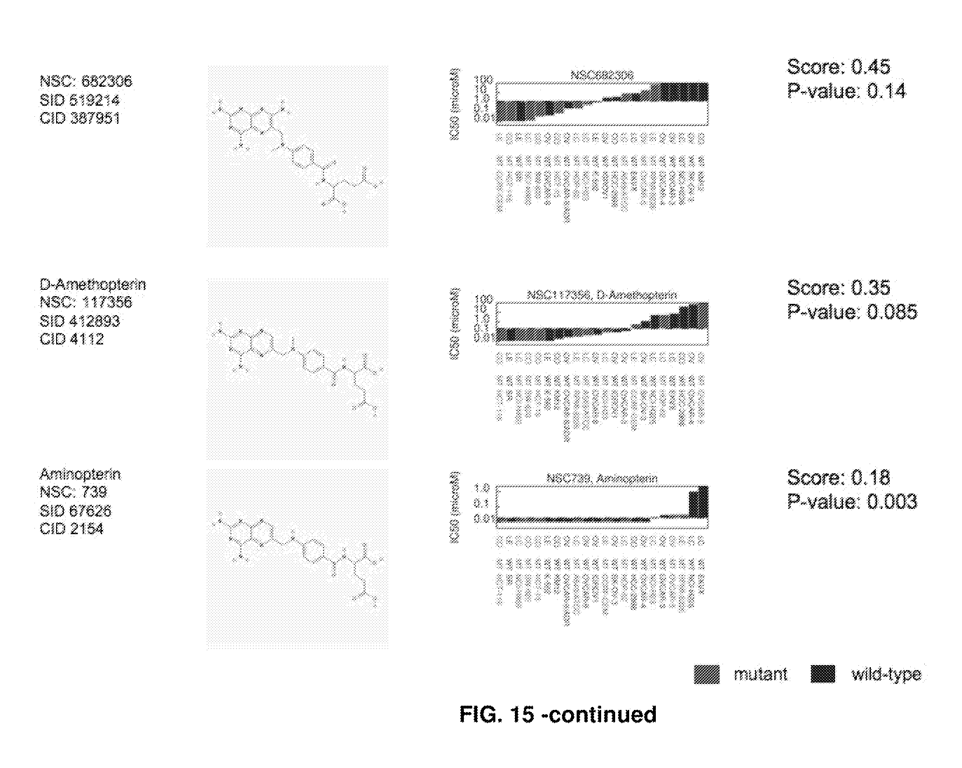

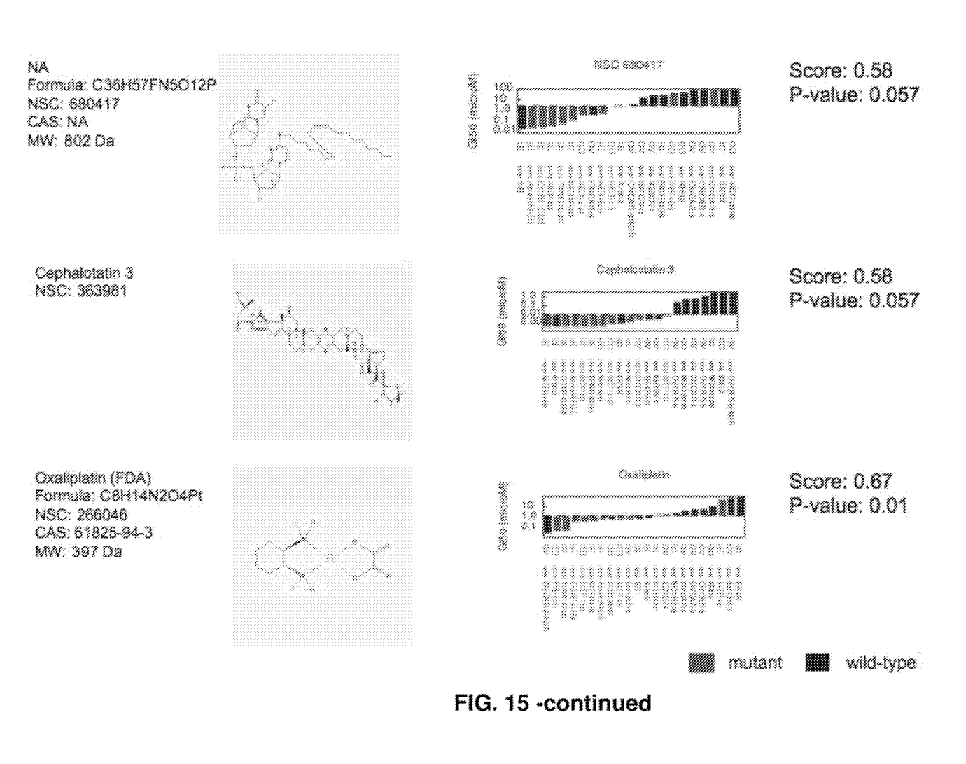

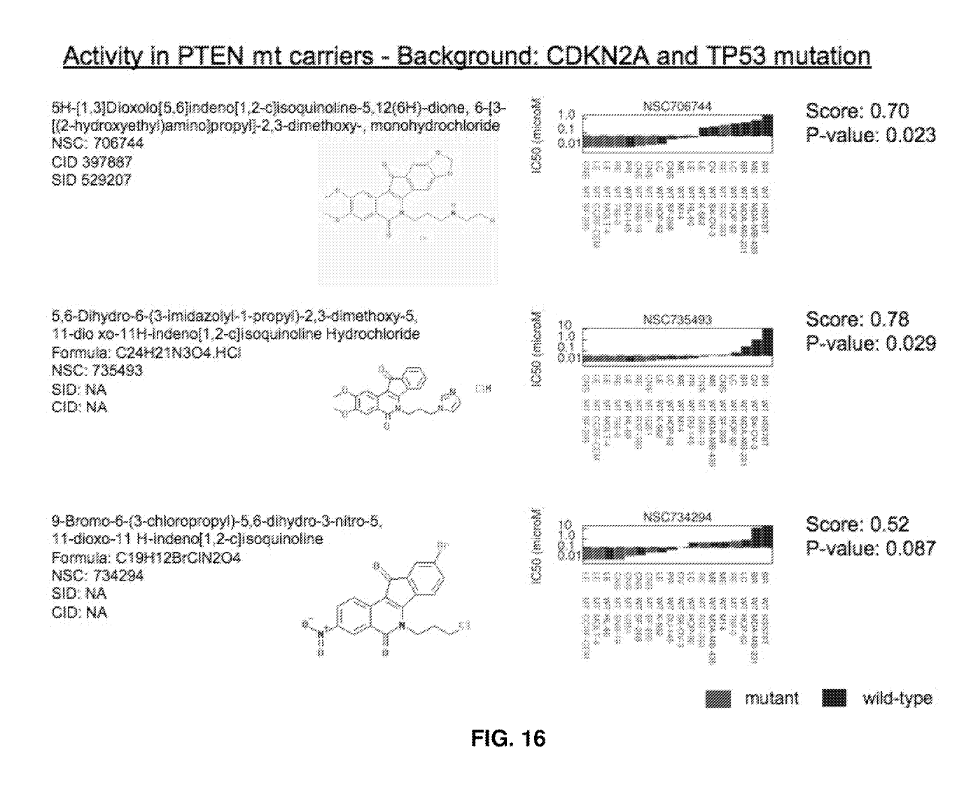

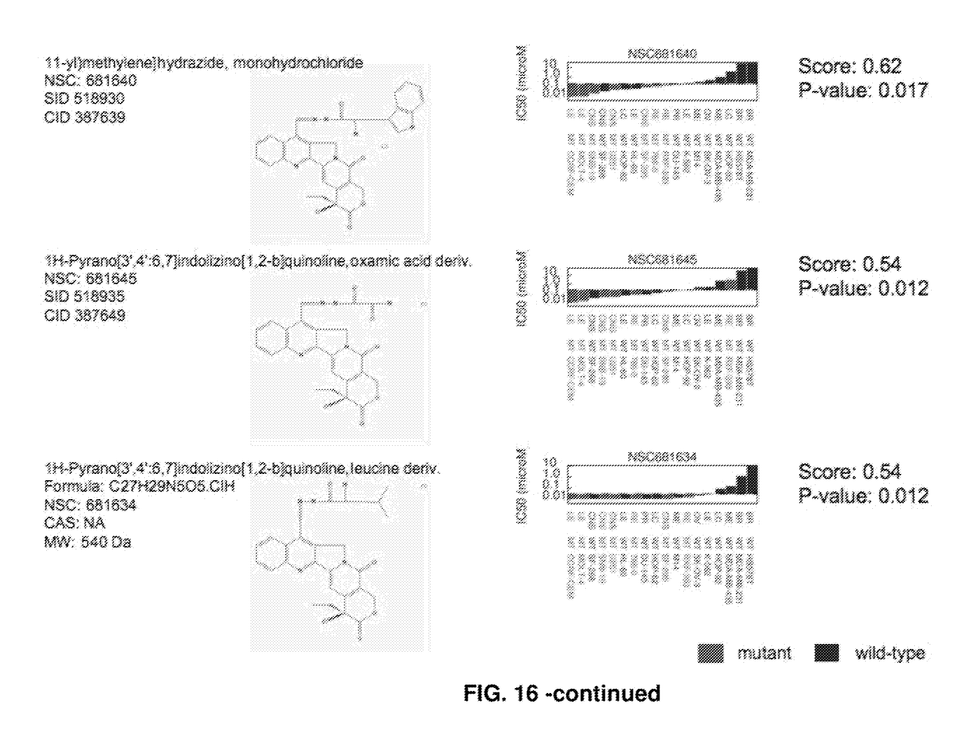

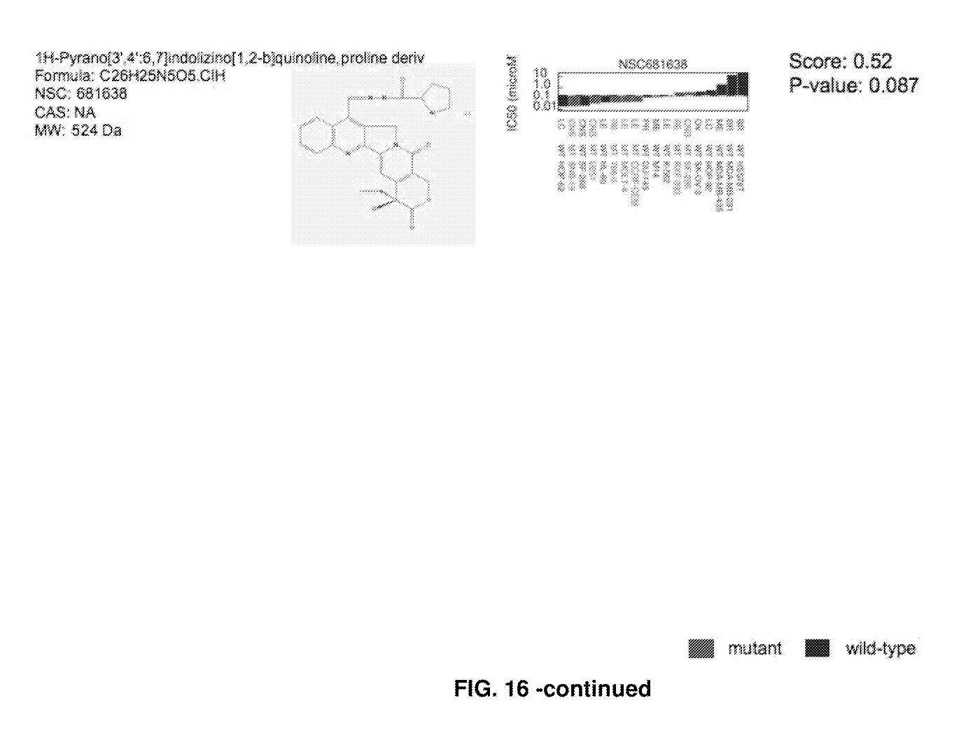

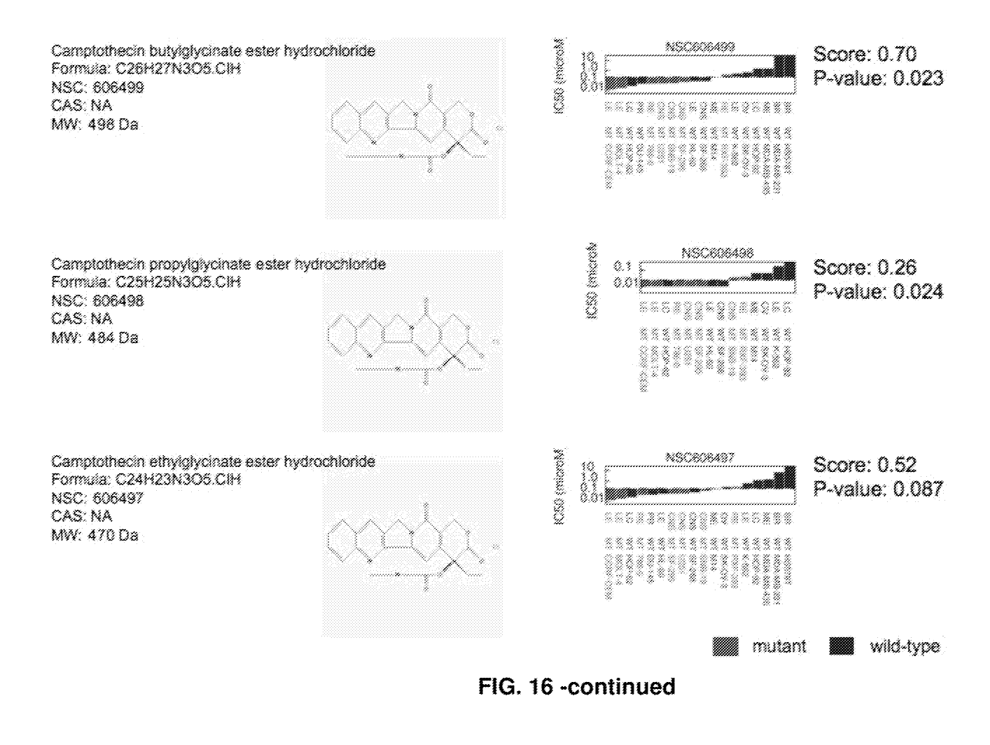

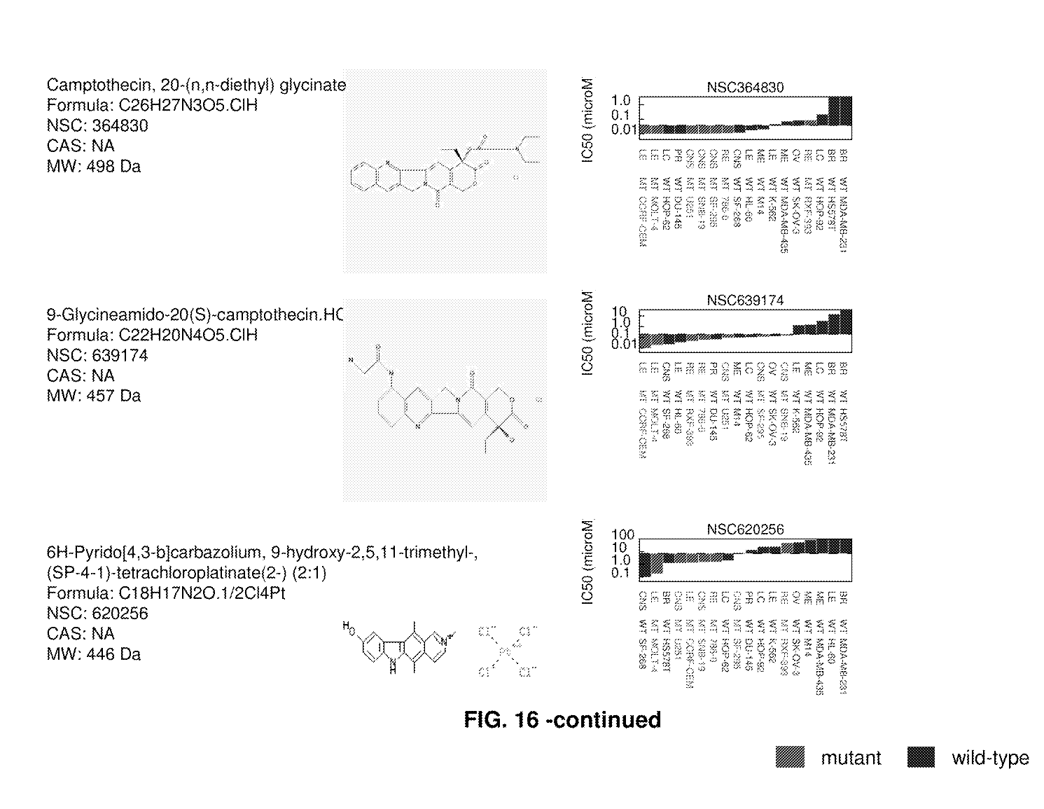

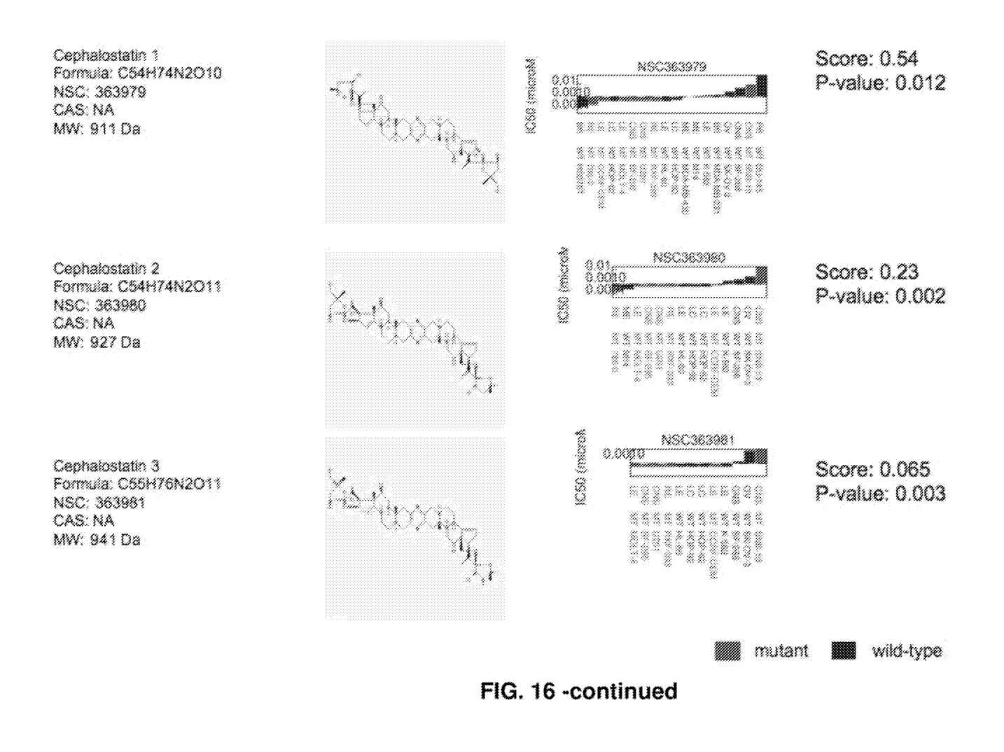

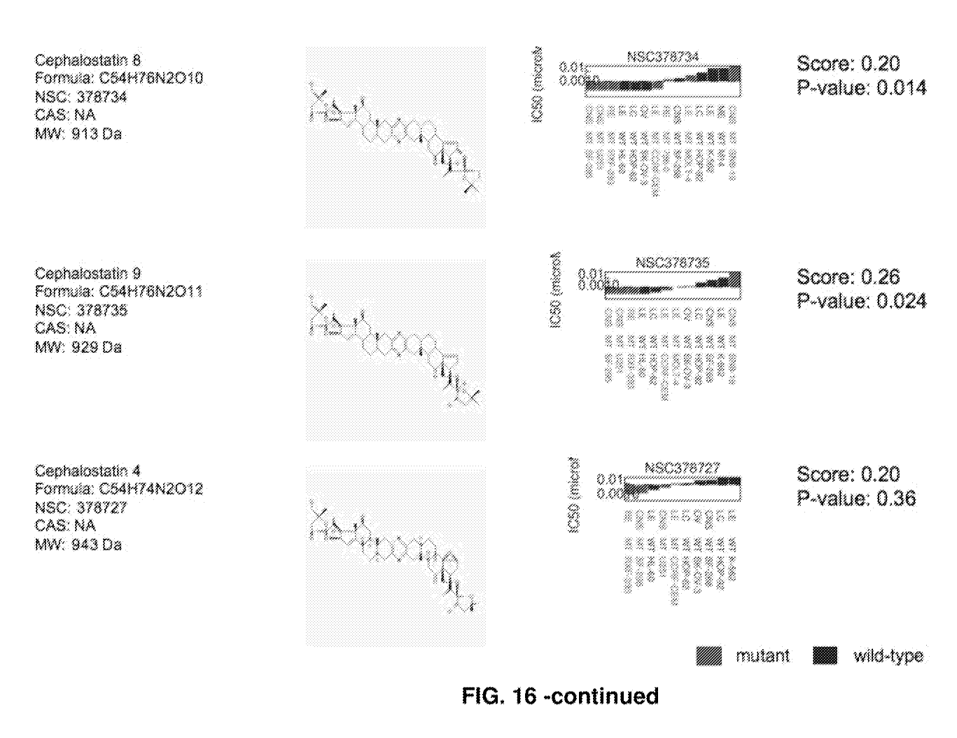

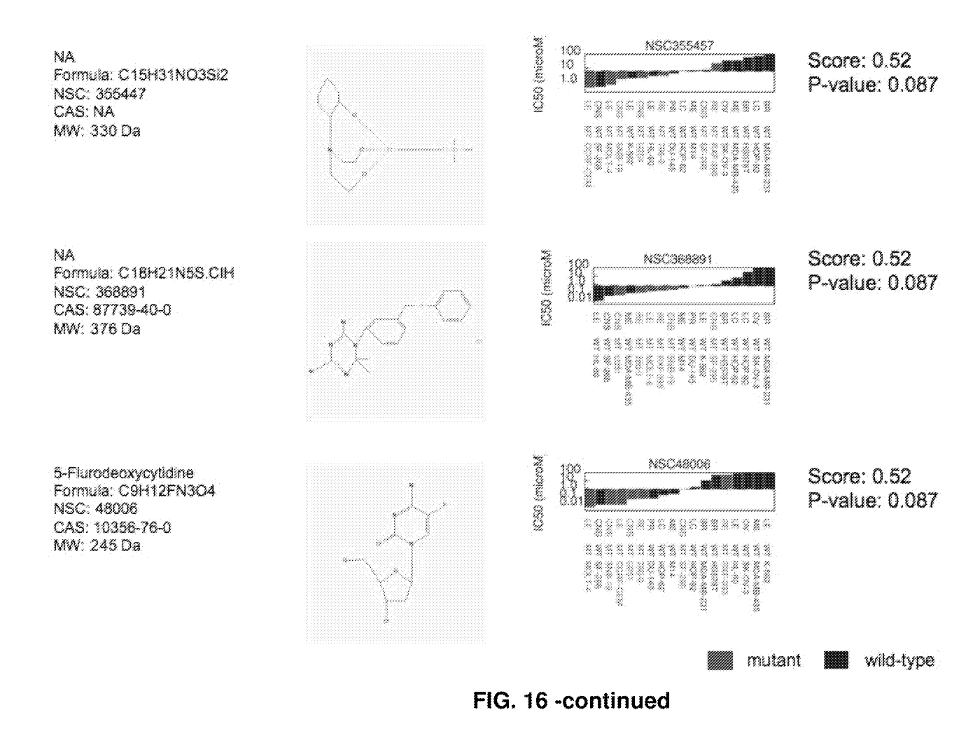

The chemical identities and structure schemes of some of the 46 compounds are shown in FIGS. 13-16.

The foregoing example and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims.

1. A method for constructing a model for determining whether a test treatment is effective for treating a disorder, comprising:
obtaining data of responsiveness of a panel of cell lines to the test treatment, wherein the panel comprises
(i) a Case Group consisting of a plurality of case cell lines each of which has a feature characterized of the disorder and
(ii) a Control Group consisting of a plurality of control cell lines each of which lacks the feature;
calculating a Responsive Fraction of the case cell lines in the Case Group that are responsive to the test treatment;
calculating a Non-Responsive Fraction of the control cell lines in the Control Group that are not responsive to the test treatment;
generating a Score of the test treatment according, to Formula I: Score=the Responsive Fraction×the Non-Responsive Fraction, or calculating statistical significance, P-value, for enrichment of responsive, cell lines in the Case Group relative to the Control group using Fischer's exact test; and
displaying a record comprising the Score or the P-value,
wherein the value of the Score indicate's effectiveness of the test treatment for treating the disorder and the P-value quantifies statistical significance.

2. The method of claim 1, wherein the test treatment is selected from the group consisting of a test compound, a microorganism, a radiation, a force, a field, a thermal energy, and a lack of a material.

3. The method of claim 2, wherein the test treatment is a test Compoundⱼ and the responsiveness of a case cell line or a control cell line in the panel to said test Compoundⱼ is obtained by a process comprising,
obtaining an IC50 value of said test Compoundⱼ against each of the cell lines in the panel of cell lines; and
calculating, as Relative Sensitivity (RS) of said test Compoundⱼ against a Cell Lineᵢ in the panel over all of the cell lines in the panel according to Formula II-1:
RSᵢⱼ=log₁₀IC50ᵢⱼ−Average(log₁₀IC50)ⱼ,
wherein
RSᵢⱼ represents the RS of said test Compound, against said Cell Lineᵢ,
IC50ᵢⱼ represents the IC50 of said test Compound, against said Cell Lineᵢ, Average(log₁₀IC50), represents the average log₁₀IC50 of said test Compoundⱼ against all of the cell lines in the panel,
i=1, 2, . . . , n,
and j=1, 2, . . . m,
wherein said Cell Lineᵢ is determined to be
(a) responsive to said test Compoundⱼ if RSᵢⱼ is less than 2σ, or
(b) non-responsive to said test Compoundⱼ if RSᵢⱼ is no less than 2σ.

4. (canceled)

5. (canceled)

6. The method of claim 2, wherein the test treatment is ionizing radiation and the responsiveness of a case cell line or a control cell line in the panel to said ionizing, radiation is obtained by a process comprising,
obtaining an ID50 value of said ionizing radiation against each of the cell lines in the panel of cell lines; and
calculating a Relative Sensitivity (RS) of said ionizing radiation against a Cell Lineᵢ in the panel over all of the cell lines in the panel according to Formula II-2:
RSᵢ=ID50ᵢ−Average(ID50),
wherein
RSᵢ represents the RS of said ionizing radiation against said Cell Lineᵢ,
ID50ᵢ represents the 11)50 of the ionizing radiation against said Cell Lineᵢ,
Average(ID50) represents the average ID50 of said ionizing radiation against all of the cell lines in the panel, and i=1, 2, . . . , n,
wherein said Cell Lineᵢ is determined to be
(a) responsive to said ionizing radiation if RSᵢ is less than 2σ, or
(b) non-responsive to said ionizing radiation if RSᵢ no less than 2σ.

7. A method of determining whether a test treatment is effective for treating, a disorder, comprising obtaining, a Score or P-value of the test treatment using the model constructed by the method of claim 1, wherein the P-value, if no larger than 0.05, or the Score, if no less than 0.5, indicates that the test treatment is effective for treating the disorder.

8. The method of claim 1, wherein the record comprises one or more of
(a) a disorder value that identities the disorder;
(b) a feature value that identities the feature characterized of the disorder;
(c) a treatment value that identifies the test treatment;
(d) a panel value that identifies the panel of cell lines;
(e) a cell value that identifies each of the cell lines in the panel;
(f) a ease group value that identifies the plurality of case cell lines;
(g) a control group value that identifies the plurality of control cell lines;
(h) a responsiveness value that identifies the responsiveness of each of the cell lines in the panel to the test treatment;
(i) a score value that identifies the Score of the test treatment; and
(j) a significance value that identifies the P-value of the test treatment.

9. (canceled)

10. The method of claim 1, wherein the disorder is a cellular proliferative disorder.

11. The method of claim 1, wherein the feature is a mutation in a gene, a gene copy alteration, overexpression or loss of a cellular gene, an alteration in a signal transduction pathway, or a resistance to a drug.

12. The method of claim 11, wherein the gene is an oncogene or a tumor suppressor gene.

13. The method of claim 12, wherein the gene is the BRAF gene, the p53 gene, the PTEN gene, or the RAS gene.

14. A machine-readable medium for carrying out the method of claim 1, comprising machine-readable instructions encoded thereon which, when executed by a processor, cause a machine having or linked to the processor to execute the method.

15. A computer system comprising the machine-readable medium of claim 14 and a user interface capable of receiving the data and displaying the record.

16. A machine-readable medium on which is stored a database capable of configuring a computer to respond to queries based on a plurality of records belonging to the database, each of the records comprising one or more of
(a) a disorder value that identities a disorder;
(b) a feature value that identifies a feature characterized of the disorder;
(c) a treatment value that identifies a test treatment for the disorder;
(d) a panel value that identifies a panel of cell lines;
(e) a cell value that identifies each of the cell lines in the panel;
(f) a case group value that identifies a plurality of case cell lines that have the feature;
(g) a control group value that identifies a plurality of control cell lines that lack the feature;
(h) a responsiveness value that identities responsiveness of each of the cell lines in the panel to the test treatment;
(i) a score value that identifies a Score of the test treatment; and
(j) a significance value that identifies at P-value of the test treatment,
wherein the Score and P value are obtained using the method of claim 1.

17. The machine-readable medium of claim 16, wherein the responsiveness value identifies the RS of a Cell Lineᵢ to a Test Treatmentⱼ, RSᵢⱼ, wherein i=1, 2, . . . , n, and j=1, 2, . . . m.

18. A computer system comprising
the machine-readable medium of claim 16 or
a user interface capable of (a) receiving a selection of one or more values for the test treatment or the cell line for determining a match between the values and a responsiveness value in the database, and (h) displaying a record associated with a matching responsiveness value.

19. (canceled)

20. (canceled)

21. A method for treating a cellular proliferative disorder in a subject, comprising administering to a subject in need thereof an effective, amount of a compound selected from the group consisting of NSC319726, NSC319725, NSC328784, NSC612941, NSC155694, NSC694266, NSC93739, NSC656238, NSC682449, NSC690432, NSC741078, NSC706829, NSC669995, NSC361127, NSC263637, NSC3541462, NSC613327, NSC146268, NSC740, NSC696558, NSC666787, NSC682306, NSC117356, NSC739, NSC680417, NSC363981, NSC266046, NSC706744, NSC735493, NSC734294, NSC681640, NSC681645, NSC681634, NSC681638, NSC606499, NSC606498, NSC606497, NSC364830, NSC639174, NSC620256, NSC363979, NSC363980, NSC363981, NSC378734, NSC378735, NSC378727, NSC355447, NSC368891, and NSC48006, or a pharmaceutically acceptable salt of the compound, wherein the cellular proliferative disorder is a condition characterized by a mutation in a gene.

22. (canceled)

23. (canceled)

24. (canceled)

25. (canceled)

26. (canceled)

27. (canceled)

28. (canceled)

29. A method for treating, a cellular proliferative disorder in a subject, characterized by a mutation in the p53 gene, comprising administering to the subject in need thereof an effective amount of a compound of Formula (Ia) or (Ib): 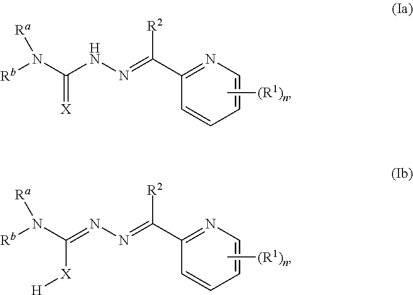
or a pharmaceutically acceptable salt of the compound, wherein:
n is 0, 1, 2, 3, or 4:
X is O, S, or Se;
R¹ at each occurrence is independently C₁-C₄ alkyl, C₁-C₄ alkoxy, hydroxyl, halogen;
R² is H, C₁-C₆ alkyl, or arylalkyl;
R^{a} and R^{b} are each independently selected from the group consisting of hydrogen, C₁-C₆ alkyl, aryl, arylalkyl, or alternatively, R^{a} and R^{b}, together with the N atom to which they are attached, form a heterocyclyl, said heterocyclyl optionally substituted by one to three substituents independently selected from C₁-C₄ alkyl, C₁-C₄ alkoxy, hydroxyl, halogen, aryl, and heteroaryl.

30. The method of claim 29, wherein;
n is 0;
R² is C₁-C₄ alkyl;
X is S or Se;
R^{a} and R^{b} are each hydrogen or C₁-C₄ alkyl, or alternatively, R^{a}R^{b}N— is heterocyclyl selected from the group consisting of;

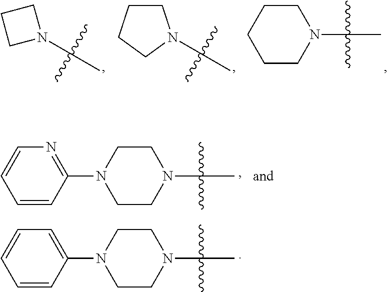

31. (canceled)

32. A method for treating a cellular proliferative disorder in a subject, characterized by a imitation in the p53 gene, comprising administering to the subject in need thereof an effective amount of a compound of Formula (II); 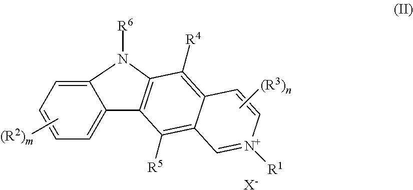
or a pharmaceutically acceptable salt of the compound, wherein:
m and n are each independently 0, 1, 2, or 3;
X⁻ is absent, halide or PtCl₄⁻;
R¹ is absent, H or C₁-C₄ alkyl;
R² at each occurrence selected from C₁-C₄ alkyl, C₁-C₄ alkoxy, R⁷C(O)—, hydroxyl, halogen;
R³, R⁴ and R⁵ are each independently selected from hydrogen, C₁-C₄ alkyl; C₁-C₄ alkoxy, R⁷C(O)—, hydroxyl, and halogen;
R⁶ is hydrogen, or C₁-C₄ alkyl; and
R⁷ is C₁-C₄ alkyl.

33. The method of claim 32, wherein:
m is 1;
n is 0:
X⁻ is iodide or PtCl₄⁻;
R¹ is methyl;
R² is hydrogen, acetyl, hydroxyl or C₁-C₄ alkoxy; and
R⁴, R₅, and R⁶ are each hydrogen or methyl.

34. (canceled)

35. A method for treating a cellular proliferative disorder in a subject, characterized by a mutation in the BRAF gene, comprising administering to the subject in need thereof an effective amount of as compound of Formula (IIIa) or (IIIb): 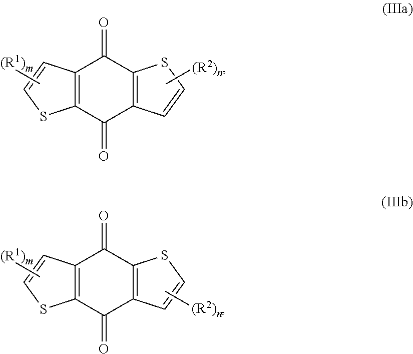
or a pharmaceutically acceptable salt of the compound, wherein:
m and n are independently 0, 1 or 2;
R¹ and R², at each occurrence, are independently selected from C₁-C₆ alkyl, halogen, hydroxyl, C alkoxy, said alkyl optionally substituted by one to three substituents independently selected from hydroxyl, halogen, and C₁-C₆ alkoxy.

36. The method of claim 35, wherein:
m and n are each independently 0 or 1; and
R¹ and R² are each independently C₁-C₄ alkyl or C₁-C₄ hydroxylalkyl.

37. (canceled)

38. A method for treating a cellular proliferative disorder in a subject, characterized by a mutation in the KRAS gene, comprising administering to the subject in need thereof an effective amount of a compound of Formula (IV): 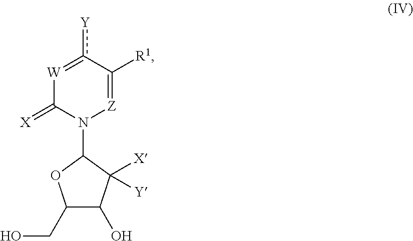
or a pharmaceutically acceptable salt of the compound, wherein;
X is O or S;
Y is NH₂ when C [symbol] Y is a single bond, or O when C [symbol] Y is a double bond;
W is NH when C [symbol] W is a single bond, or N when C [symbol] W is a double bond;
Z is N or CH;
X′ and Y′ are each independently H, hydroxyl or halogen; and
R¹ is 11 or halogen.

39. (canceled)

40. A method for treating a cellular proliferative disorder in a subject, characterized by a mutation in the KRAS gene, comprising administering to the subject in need thereof an effective amount of as compound of Formula (V): 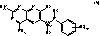
or a pharmaceutically acceptable salt of the compound, wherein:
m is 0, 1, 2, 3, 4, or 5
n is 1,2, or 3;
Z is N or C—R^{z}, wherein R^{z} is H or C₁-C₄ alkyl;
R¹, at each occurrence, is independently selected from halogen, hydroxyl, C₁-C₄ alkoxy, and R⁵—C(O)—;
R² is hydrogen or C₁-C₄ alkyl;
R³, at each occurrence, is independently hydrogen, C₁-C₄ alkyl, halogen or hydroxyl;
R⁴ is hydrogen, halogen, hydroxyl, or —NH₂;
R⁵ is hydrogen, C₁-C₆ alkyl, or —NR^{a}R^{b};
R^{a} and R^{b} are each independently selected from hydrogen, benzyl, and C₁-C₆ alkyl optionally substituted by one, two, or three substituents independently selected from halogen, hydroxyl, —CO₂R⁶, and —SO₃R⁶; and
R⁶ is hydrogen or C₁-C₄ alkyl.

41. The method of claim 40, wherein;
m is 1, 2 or 3;
n is 1;
Z is N or C—CH₃;
R¹ is halogen or R⁵—C(O)—;
R² is hydrogen or methyl:
R³ is hydrogen;
R⁴ is hydrogen or —NH₂;
R⁵ is hydrogen, C₁-C₆ alkyl, or —NHR^{a};
R^{a} is C₁-C₆ alkyl optionally substituted by one, or two substituents independently selected from —CO₂R⁶, and —SO₃R⁶; and
R⁶ is hydrogen or C₁-C₄ alkyl.

42. (canceled)

43. A method for treating a cellular proliferative disorder in a subject, characterized by a mutation in the PTEN gene, comprising administering, to the subject in need thereof an effective amount of it compound of Formula (VI): 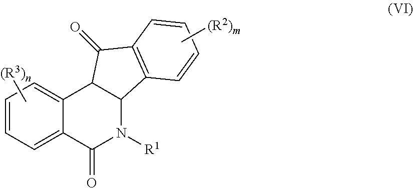
or a pharmaceutically acceptable salt of the compound, wherein:
m and n are each independently 0, 1, 2, 3, or 4.
R¹ is selected from hydrogen, C₁-C₆ alkyl optionally substituted by one two or three substituents independently selected from the group consisting of hydroxyl, halogen, aryl, heteroaryl, heterocyclyl, and —NR^{a}R^{h};
R² and le, at each occurrence, are independently selected from hydrogen, halogen, hydroxyl, C₁-C₄ alkoxy, C₁-C₄ haloalkoxy, nitro, aryl, and heterocyclyl, or alternatively, two R² or two R³ combined form a five- or six-membered heterocyclyl ring fused onto the respective benzene ring of the molecule; and
R^{a} and R^{b} are each independently selected from hydrogen. C₁-C₄ alkyl optionally substituted by one or two substituents independently selected from halogen, hydroxyl, and C₁-C₄ alkoxy.

44. The method of claim 43, wherein;
m is 1 or 2;
n is 1 or 2;
R¹ is —(CH₂)ᵢ—R^{x}, wherein i is 2, 3, or 4, and R^{x} is halogen, hydroxyl, heterocyclyl, or substituted alkylamino;
R² is hydrogen, halogen, C₁-C₄ alkoxy, or alternatively, two of R²'s together form a five member-heterocyclyl ring fused onto the benzene ring to which R² is attached; and
R³ at each occurrence is independently selected from C₁-C₄ alkoxy and nitro.

45. (canceled)

46. A method for treating a cellular proliferative disorder in a subject, characterized by a mutation in the PTEN gene, comprising administering to the subject in need thereof an effective amount of a compound of Formula (VII): 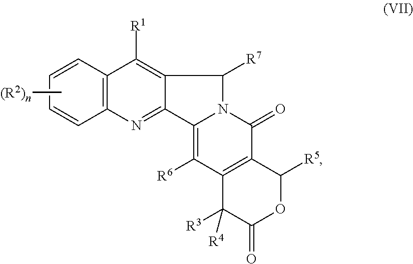
or a pharmaceutically acceptable salt of the compound, wherein:
n is 0, 1, 2, 3, or 4:
R¹ is selected from hydrogen, C₁-C₆ alkyl, hydroxyl, C₁-C₆ alkoxy, halogen, and a group characterized by formula (A): 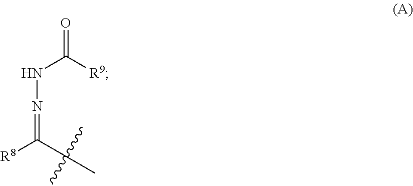
R² at each occurrence is independently selected from hydrogen, C₁-C₆ alkyl, hydroxyl, C₁-C₆ alkoxy, halogen, and —NR^{a}R^{b}:
R³ and R⁴ are each independently hydrogen, C₁-C₆, alkyl, hydroxyl, C₁-C₆ alkoxy, R¹²C(O)O—, halogen, and —NR^{a}R^{b};
R⁵ is hydrogen or C₁-C₄ alkyl;
R⁶ is hydrogen, C₁-C₆ alkyl, hydroxyl, C₁-C₆ alkoxy, or halogen;
R⁷ is hydrogen or C₁-C₄ alkyl;
R^{a} and R^{b} are independently hydrogen, C₁-C₄ alkyl, and R¹⁰C(O)—;
R⁸ is hydrogen or C₁-C₄ alkyl:
R⁹ is selected from C₁-C₆ alkyl, aryl, heteroaryl, heterocyclyl, —C(O)NR^{c}R^{d}, wherein said alkyl is optionally substituted by one or two substituents independently selected from the group consisting of hydroxyl, halogen, C₁-C₄ alkoxy, —SR¹¹, aryl, heteroaryl, heterocyclyl, and —NR^{c}R^{d};
R¹⁰ is C₁-C₄ alkyl or NR^{c}R^{d};
R¹¹ is hydrogen or C₁-C₄ alkyl;
R¹² is C₁-C₆ alkyl optionally substituted by —NR^{c}R^{d}; and
R^{c} and R^{d} are each independently hydrogen or C₁-C₆ alkyl.

47. The method of claim 46, wherein:
n is 0 or 1;
R¹ is hydrogen or a group of formula (A): 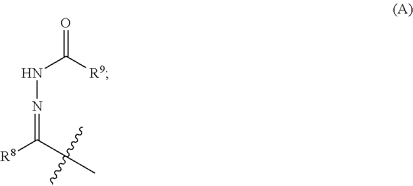
R² is —NHR^{a};
R³ is C₁-C₆ alkyl;
R⁴ is hydroxyl or R¹²C(O)O—;
R⁵ hydrogen;
R⁶ is hydrogen;
R⁷ is hydrogen;
R₈ is hydrogen;
R⁹ is selected from heterocyclyl, —C(O)NH₂, and C₁-C₆ alkyl optionally substituted by one or two substituents independently selected from the group consisting of hydroxyl; halogen, C₁-C₄ alkoxy, —SR¹¹, aryl, heteroaryl, heterocyclyl, and —NH₂;
R^{a} is hydrogen or R¹⁰C(O)—;
R¹⁰ at each occurrence is independently C₁-C₄ alkyl or NH₂;
R¹¹ is hydrogen or C₁-C₄ alkyl; and
R¹² is NHR^{c}.

48. (canceled)

49. A method for treating a cellular proliferative disorder in a subject, characterized by a mutation in the PTEN gene, comprising administering to the subject in need thereof an effective amount of a compound of Formula (VIII): 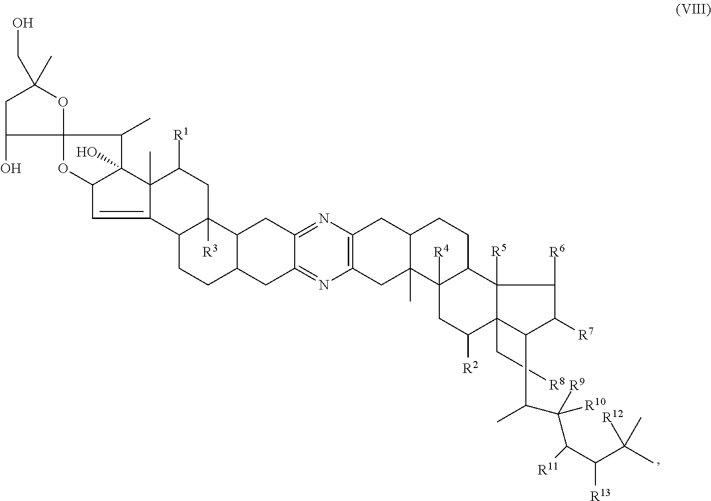
or a pharmaceutically acceptable salt of the compound, wherein:
R¹ and R² are each independently OH or oxo (═O);
R³ and R⁴ are each independently H or OH;
R⁵ and R⁶ together form a bond or an epoxide (—O—);
R⁷ is H or OH, or alternatively in combination with R⁹ or R¹⁰ forms an oxygen bridge (—O—);
R⁸ is OH or alternatively in combination with R⁹ or R¹⁰ forms an oxygen bridge (—O—);
R⁹ and R¹⁰ are each OH, or alternatively in combination with R⁷, R⁸ or R¹² form an oxygen bridge (—O—);
R¹¹ is H or OH;
R¹² OH, or alternatively in combination with R⁹ or R¹⁰ forms an oxygen bridge (—O—); and
R¹³ is H or CH₃.

50. The method of claim 49, wherein said compound is one of cephalostatins.

51. (canceled)

52. A method for treating a cellular proliferative disorder in a subject, comprising administering to the subject in need thereof a composition comprising an effective amount of NSC319726 or pharmaceutically acceptable salt thereof.

53. (canceled)

54. (canceled)

55. (canceled)

56. (canceled)

57. (canceled)
